(19) United States
(12) Patent Application Publication
(10) Pub. No.: US 2024/0252062 A1
(43) Pub. Date: Aug. 1, 2024

(54) EAR HEALTH CONDITION DETERMINATION

(71) Applicant: QUALCOMM Incorporated, San Diego, CA (US)

(72) Inventors: Ritesh GARG, Hyderabad (IN); Vishnu Vardhan KASILYA SUDARSAN, Bangalore (IN); Pruthvi Raj SINGH, Hyderabad (IN); Sumeet Kumar SAHU, Berhampur (IN)

(21) Appl. No.: 18/160,849

(22) Filed: Jan. 27, 2023

(51) Int. Cl. A61B 5/12; A61B 5/00

(52) U.S. Cl. CPC A61B 5/125; A61B 5/725; A61B 5/7264; A61B 2562/0204

(57) ABSTRACT

A device includes a processor configured to receive a signal from a feedback microphone of an earphone. The processor is also configured to determine, based on a change over time of sound reflection characteristics represented in the received signal, a condition corresponding to ear wax buildup or water in an ear canal.

### I. FIELD

The present disclosure is generally related to determining a condition related to ear health.

### II. DESCRIPTION OF RELATED ART

Ear wax, also known as cerumen, is a sticky and waxy substance that is naturally produced by glands in the ear canal. Ear wax serves to trap dirt and debris, preventing such contaminants from travelling deep inside the ear canal. Ear wax also lubricates the ear canal and helps slow the growth of bacteria. Ear wax typically dries up and falls out of the ear, which can be assisted by jaw movement and chewing motions that help move excess ear wax towards the entrance of the ear.

In some circumstances, ear wax can build up in the ear canal, which can cause a gradual diminishment in a person's hearing and an increased likelihood of infection which, if left untreated, could cause permanent hearing loss. Ear wax blockage can result from impaction due to inserting an object into the ear canal, such as by the improper use of a cotton swab. Additionally, it has also been theorized that use of ear plugs or in-ear style earphones for long periods of time may interfere with the natural removal of ear wax. For example, when an ear plug is worn, ear wax may be prevented from leaving the ear and may instead be pushed into the ear by the ear plug.

Since ear wax buildup is a gradual process that can occur over relatively long time spans, in most cases people experiencing ear wax buildup are not able to perceive, or do not notice, the gradual diminishment of hearing that results from ear wax buildup. People may therefore remain unaware of their ear health condition until an ear wax blockage of the ear canal results in pressure or pain, or when infection occurs. Providing people with the ability to detect ear health conditions such as ear wax buildup prior to the occurrence of acute symptoms would enable them to perform remedial actions, such as ear cleaning or medical intervention, resulting in improved ear health and hearing, as well as an enhanced experience when using earphone devices.

### III. SUMMARY

According to a particular aspect, a device includes a processor configured to receive a signal from a feedback microphone of an earphone. The processor is also configured to determine, based on a change over time of sound reflection characteristics represented in the received signal, a condition corresponding to ear wax buildup or water in an ear canal.

According to a particular aspect, a method includes receiving, at a processor, a signal from a feedback microphone of an earphone. The method also includes determining, based on a change over time of sound reflection characteristics represented in the received signal, a condition corresponding to ear wax buildup or water in an ear canal.

According to a particular aspect, a non-transitory computer-readable medium stores instructions that, when executed by one or more processors, cause the one or more processors to receive a signal from a feedback microphone of an earphone. The instructions, when executed by the one or more processors, also cause the one or more processors to determine, based on a change over time of sound reflection characteristics represented in the received signal, a condition corresponding to ear wax buildup or water in an ear canal.

Other aspects, advantages, and features of the present disclosure will become apparent after review of the entire application, including the following sections: Brief Description of the Drawings, Detailed Description, and the Claims.

### IV. BRIEF DESCRIPTION OF THE DRAWINGS

FIG. 1 is a block diagram of a particular illustrative aspect of a system operable to determine an ear health condition, in accordance with some examples of the present disclosure.

FIG. 2 is a diagram of particular aspects of the system of FIG. 1, in accordance with some examples of the present disclosure.

FIG. 3 is a diagram of particular aspects of the system of FIG. 1, in accordance with some examples of the present disclosure.

FIG. 4 is a diagram of particular aspects of the system of FIG. 1, in accordance with some examples of the present disclosure.

FIG. 5 is a diagram of particular aspects of the system of FIG. 1, in accordance with some examples of the present disclosure.

FIG. 6 is a diagram of particular aspects of the system of FIG. 1, in accordance with some examples of the present disclosure.

FIG. 7 illustrates an example of an integrated circuit operable to determine an ear health condition, in accordance with some examples of the present disclosure.

FIG. 8 is a diagram of earbuds operable to determine an ear health condition, in accordance with some examples of the present disclosure.

FIG. 9 is a diagram of a headset operable to determine an ear health condition, in accordance with some examples of the present disclosure.

FIG. 10 is a diagram of a headset, such as a virtual reality, mixed reality, or augmented reality headset, operable to determine an ear health condition, in accordance with some examples of the present disclosure.

FIG. 11 is a diagram of a system including a mobile device operable to determine an ear health condition, in accordance with some examples of the present disclosure.

FIG. 12 is a diagram of a system including a wearable electronic device operable to determine an ear health condition, in accordance with some examples of the present disclosure.

FIG. 13 is a diagram of a voice-controlled speaker system operable to determine an ear health condition, in accordance with some examples of the present disclosure.

FIG. 14 is a diagram of an example of a vehicle operable to determine an ear health condition, in accordance with some examples of the present disclosure.

FIG. 15 is a diagram of a particular implementation of a method of determining an ear health condition that may be performed by the device of FIG. 1, in accordance with some examples of the present disclosure.

FIG. 16 is a diagram of a particular implementation of a method of determining an ear health condition that may be performed by the device of FIG. 1, in accordance with some examples of the present disclosure.

FIG. 17 is a diagram of a particular implementation of a method of determining an ear health condition that may be performed by the device of FIG. 1, in accordance with some examples of the present disclosure.

FIG. 18 is a block diagram of a particular illustrative example of a device that is operable to determine an ear health condition, in accordance with some examples of the present disclosure.

### V. DETAILED DESCRIPTION

Ear wax blockage can result from impaction due to inserting an object into the ear canal, and may also result from gradual buildup of ear wax over time when the ear wax is prevented from exiting the ear. However, since ear wax buildup is a gradual process that can occur over relatively long time spans, in most cases people experiencing ear wax buildup are not able to perceive, or do not notice, the gradual diminishment of hearing that accompanies ear wax buildup. Such people may remain unaware of their ear health condition until an ear wax blockage of the ear canal results in pressure or pain, or when infection occurs.

Systems and methods of detecting an ear health condition are described. For example, according to a particular aspect, sound reflections from an eardrum can be monitored using a feedback microphone of an earphone, and a change over time of the sound reflection characteristics can indicate an ear health condition such as ear wax buildup. The wearer of the earphone is notified of the ear health condition, enabling the wearer to perform remedial actions, such as ear cleaning or medical intervention, prior to the occurrence of acute symptoms. Thus, the disclosed systems and methods enable users to have improved ear health and hearing, as well as an enhanced experience when using the earphone.

According to some aspects, signal processing components of an active noise cancellation (ANC) processor, such as a digital signal processor (DSP), of an earphone are used to monitor sound reflections from within the ear to determine a change of ear wax buildup over time. The earphone can produce the sound in a calibration operation or during normal use of the earphone. The sound is reflected from the eardrum or from one or more obstructions in the ear canal, and the reflections are captured by a feedback microphone of the earphone and processed at the ANC components of the earphone.

According to some aspects, the output of the feedback microphone is processed by one or more adaptive filters of the ANC to generate a set of adaptive filter weights. The adaptive filter weights are determined so that, when used to filter the output sound signal, the resulting filtered signal substantially matches the output of the feedback microphone. Thus, the adaptive filter weights can be representative of an impulse response associated with the reflection path from the earphone speaker to the eardrum and back to the feedback microphone. In particular, the adaptive filter weights may exhibit a peak having a height that indicates the strength of the reflected signal and having a filter tap position that indicates a length of the reflection path.

Changes in the peak over time can be used to determine one or more ear health conditions. For example, a gradual reduction of the peak height over time can indicate ear wax buildup, and the system may notify the user when the peak height has fallen below a threshold. According to some aspects, one or more other ear health conditions may be determined. For example, a sudden decline in the peak height can indicate swelling of the ear canal, such as due to infection, and detection of a sudden decline of the peak height can trigger a notification to the user. As another example, the user may be notified in response to detecting an increase in the peak height, which can indicate the presence of water or other fluid or obstruction in the ear canal. In another example, the user may be notified in response to detecting a change in the tap position of the peak to a position associated with a shorter reflection path, which can indicate that an ear tip of the earphones has become obstructed.

In some implementations, the reflected signal analysis and ear health condition monitoring is performed within the earphone, such as by a processor integrated in an in-ear, on-ear, or over-ear earphone device. In such cases, the user may be notified of a detected ear health condition via an audio signal played out via the earphones, such as a voice message. In other implementations, the reflected signal analysis and ear health condition monitoring is performed by a device that is coupled to the earphone, such as a mobile phone that is wirelessly coupled to a pair of earbuds. In such cases, a display or other user interface of the device may be used to notify the user of a detected ear health condition instead of, or in addition to, an audible notification.

By automatically monitoring for ear health conditions during normal operation of the earphones and notifying the user when an ear health condition is determined, the disclosed systems and methods enable the wearer to initiate ear cleaning or seek medical intervention at an early stage and potentially prior to the user noticing any symptoms of the ear health condition. Thus, the disclosed systems and methods enable users to have improved ear health and hearing, as well as an enhanced experience when using the earphone.

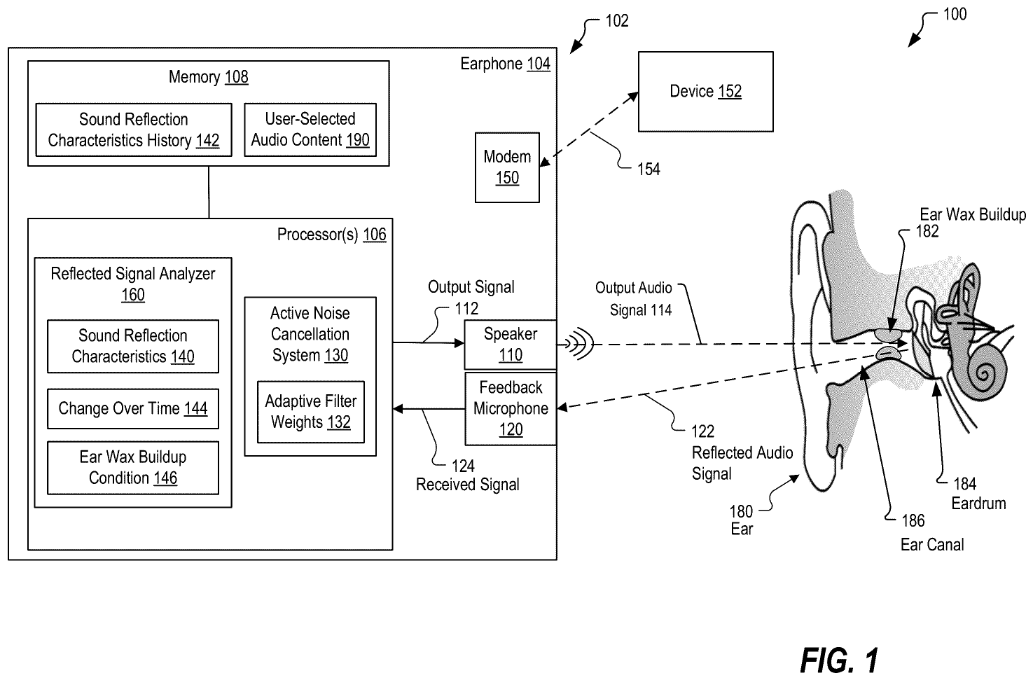

Particular aspects of the present disclosure are described below with reference to the drawings. In the description, common features are designated by common reference numbers. As used herein, various terminology is used for the purpose of describing particular implementations only and is not intended to be limiting of implementations. For example, the singular forms “a,” “an,” and “the” are intended to include the plural forms as well, unless the context clearly indicates otherwise. Further, some features described herein are singular in some implementations and plural in other implementations. To illustrate, FIG. 1 depicts a device 102 including one or more processors (“processor(s)” 106 of FIG. 1), which indicates that in some implementations the device 102 includes a single processor 106 and in other implementations the device 102 includes multiple processors 106. For ease of reference herein, such features are generally introduced as “one or more” features and are subsequently referred to in the singular or optional plural (as indicated by “(s)” in the name of the feature) unless aspects related to multiple of the features are being described.

As used herein, the terms “comprise,” “comprises,” and “comprising” may be used interchangeably with “include,” “includes,” or “including.” Additionally, the term “wherein” may be used interchangeably with “where.” As used herein, “exemplary” indicates an example, an implementation, and/or an aspect, and should not be construed as limiting or as indicating a preference or a preferred implementation. As used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). As used herein, the term “set” refers to one or more of a particular element, and the term “plurality” refers to multiple (e.g., two or more) of a particular element.

As used herein, “coupled” may include “communicatively coupled,” “electrically coupled,” or “physically coupled,” and may also (or alternatively) include any combinations thereof. Two devices (or components) may be coupled (e.g., communicatively coupled, electrically coupled, or physically coupled) directly or indirectly via one or more other devices, components, wires, buses, networks (e.g., a wired network, a wireless network, or a combination thereof), etc. Two devices (or components) that are electrically coupled may be included in the same device or in different devices and may be connected via electronics, one or more connectors, or inductive coupling, as illustrative, non-limiting examples. In some implementations, two devices (or components) that are communicatively coupled, such as in electrical communication, may send and receive signals (e.g., digital signals or analog signals) directly or indirectly, via one or more wires, buses, networks, etc. As used herein, “directly coupled” may include two devices that are coupled (e.g., communicatively coupled, electrically coupled, or physically coupled) without intervening components.

In the present disclosure, terms such as “determining,” “calculating,” “estimating,” “shifting,” “adjusting,” etc. may be used to describe how one or more operations are performed. It should be noted that such terms are not to be construed as limiting and other techniques may be utilized to perform similar operations. Additionally, as referred to herein, “generating,” “calculating,” “estimating,” “using,” “selecting,” “accessing,” and “determining” may be used interchangeably. For example, “generating,” “calculating,” “estimating,” or “determining” a parameter (or a signal) may refer to actively generating, estimating, calculating, or determining the parameter (or the signal) or may refer to using, selecting, or accessing the parameter (or signal) that is already generated, such as by another component or device.

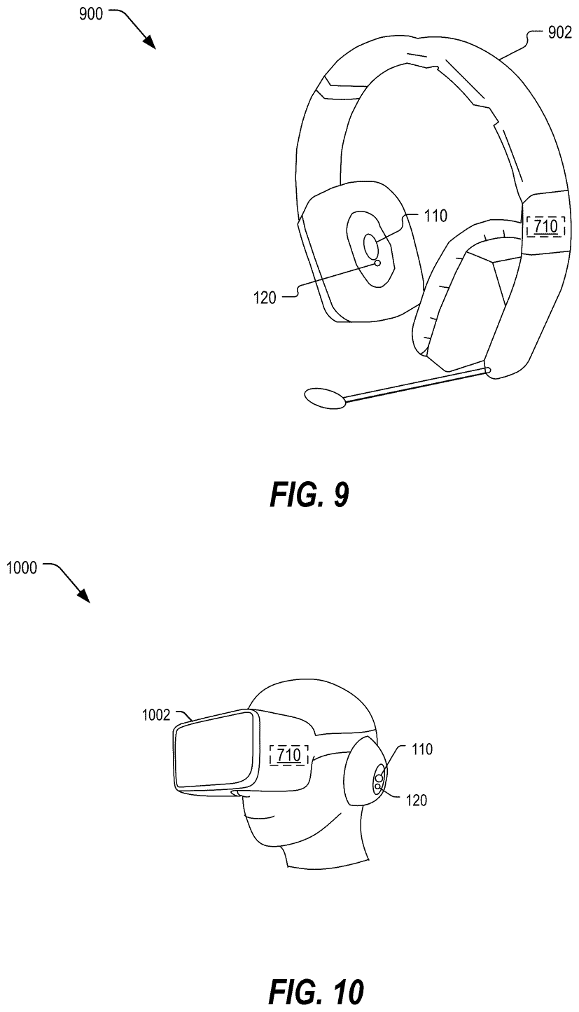

Referring to FIG. 1, a particular illustrative aspect of a system 100 configured to determine an ear health condition is shown. In the example illustrated in FIG. 1, the system 100 includes a device 102 configured to determine an ear health condition based on a signal 124 received from a feedback microphone 120 of an earphone 104. In some implementations, the earphone 104 corresponds to an in-ear style earphone, such as earbuds illustrated in FIG. 8. In other implementations, the earphone 104 corresponds to an on-ear or over-ear style earphone, such as illustrated in FIG. 9.

The device 102 includes one or more processors 106 coupled to a memory 108, a speaker 110 configured to generate an output audio signal 114, and the feedback microphone 120. The one or more processors 106 include a DSP, one or more other types of processor, or a combination thereof. The one or more processors 106 are configured to receive the signal 124 from the feedback microphone 120 and to determine, based on a change over time 144 of sound reflection characteristics 140 represented in the received signal 124, a condition 146 corresponding to ear wax buildup 182.

To illustrate, the one or more processors 106 are configured to send an output signal 112 to the speaker 110, which plays out a corresponding output audio signal 114. The output audio signal 114 propagates along an ear canal 186 to the eardrum 184 of a user's ear 180. A portion of the output audio signal 114 is reflected from the eardrum 184 as a reflected audio signal 122, which is captured by the feedback microphone 120 and represented as a component of the received signal 124.

The one or more processors 106 include a reflected signal analyzer 160 configured to analyze the sound reflection characteristics 140 of the reflected audio signal 122 to determine the ear wax buildup condition 146. In particular, because the ear wax buildup 182 attenuates the output audio signal 114 and the reflected audio signal 122, the magnitude, or energy, of the corresponding component of the received signal 124 is reduced as compared to when there is no (or a reduced amount of) ear wax buildup. The ear wax buildup 182 can also delay the output audio signal 114 and the reflected audio signal 122 due to slower propagation through the ear wax, resulting in a delay, temporal spreading, or both, of the reflected audio signal 122 reaching the feedback microphone 120 and therefore of the corresponding component of the signal 124 received at the one or more processors 106.

The reflected signal analyzer 160 determines the amount of attenuation (and, in some implementations, the amount of delay or temporal spreading) in the received signal 124 as part of the sound reflection characteristics 140. The reflected signal analyzer 160 can compare the sound reflection characteristics 140 to baseline data stored in a sound reflection characteristics history 142 in the memory 108 to determine the change over time 144. For example, the sound reflection characteristics history 142 may include calibration data generated after an ear cleaning, sound reflection characteristics from one or more other previous ear health detection operations, or a combination thereof. In some implementations, the reflected signal analyzer 160 compares a characteristic of the received signal 124 to a threshold value that is based on one or more previous measurements to detect the ear wax buildup condition 146.

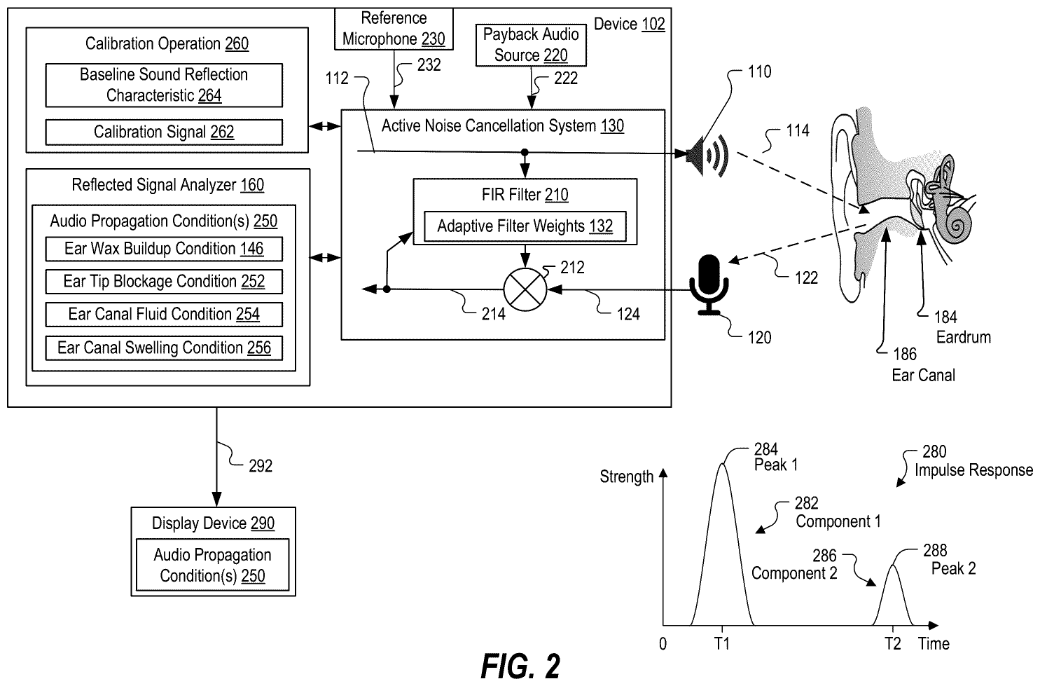

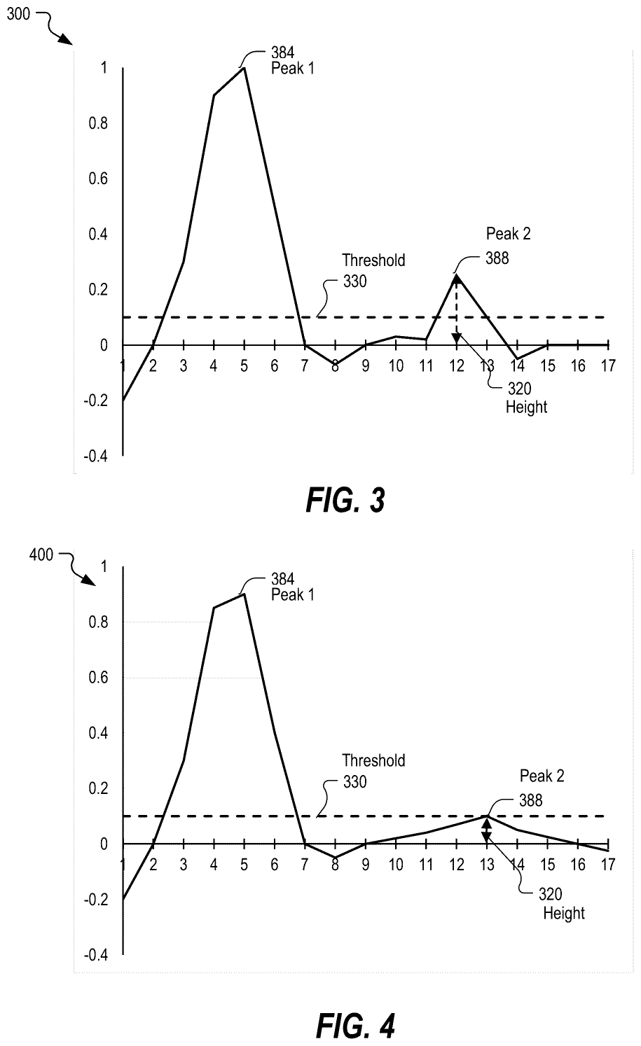

According to some aspects, the one or more processors 106 are configured to track the sound reflection characteristics 140 over time based on adaptive filter weights 132 of an active noise cancellation system 130. To illustrate, the signal 124 received from the feedback microphone 120 includes a component corresponding to a reflection of the output audio signal 114 from the eardrum 184 (e.g., the reflected audio signal 122) and, as described further with reference to FIG. 2, the sound reflection characteristics 140 can correspond to a height of a peak that is associated with the adaptive filter weights 132 and that corresponds to the reflection from the eardrum 184. In a particular example, the reflected signal analyzer 160 is configured to determine the condition 146 corresponding to ear wax buildup based on the height of the peak falling below a threshold, such as described further with reference to FIG. 3 and FIG. 4.

During operation, the device 102 may initiate a calibration operation to determine a baseline sound reflection characteristic, such as when a user of the device 102 has recently had an ear cleaning and instructs the device 102, via a user interface (e.g., a touch-sensitive user interface or a voice interface) to perform the calibration operation. In a particular implementation, during the calibration operation, the device 102 (e.g., the one or more processors 106) is configured to initiate playback of a calibration signal into the ear canal 186. For example, the one or more processors 106 may send a particular output signal 112 to the speaker 110 having particular frequency characteristics, such as having signal components primarily (or exclusively) in the frequency range of 50-250 hertz (Hz). To illustrate, the calibration signal can be a single tone at 150 Hz. Based on the resulting received signal 124 associated with the calibration signal, the one or more processors 106 (e.g., the active noise cancellation system 130) can estimate an impulse response of the ear canal. The reflected signal analyzer 160 can determine a baseline sound reflection characteristic based on a height of a peak associated with the impulse response and corresponding to reflection from the eardrum 184. The baseline sound reflection characteristic, data such as one or more thresholds generated based on the baseline sound reflection characteristic, or a combination thereof, can be saved in the sound reflection characteristics history 142.

After calibration, the device 102 can track the sound reflection characteristics 140 by periodically or occasionally performing an ear health determination operation to determine the change over time 144 of the sound reflection characteristics 140. For example, the reflected signal analyzer 160 can be configured to determine updated sound reflection characteristics 140 during normal operation of the device 102, such as during playback of user-selected audio content 190 at the earphone 104, and according to a stored ear health evaluation schedule or based on one or more other criteria.

According to some aspects, the active noise cancellation system 130 processes the received signal 124 to determine the adaptive filter weights 132 associated with the received signal 124, as described further below with reference to FIGS. 2-5, and the reflected signal analyzer 160 determines the sound reflection characteristics 140 based on a peak in the adaptive filter weights 132 that is associated with the reflected audio signal 122. The reflected signal analyzer 160 processes the sound reflection characteristics 140 to determine the change over time 144, such as a change in the peak as compared to during calibration.

In some implementations, the reflected signal analyzer 160 compares the height of the peak of the adaptive filter weights 132 that is associated with the reflected audio signal 122 to a threshold peak height that is extracted from the sound reflection characteristics history 142. For example, the threshold peak height may be determined as a percentage (e.g., 60%) of a calibration peak height. In response to the peak height associated with the reflected audio signal 122 being lower than the threshold peak height, the reflected signal analyzer 160 determines that the ear wax buildup 182 has caused sufficient attenuation in the reflected audio signal 122 to indicate the ear wax buildup condition 146.

Although the device 102 is described in conjunction with determining the ear wax buildup condition 146, in other implementations the device 102 is configured to process the received signal 124 to determine an audio propagation condition associated with one or more other ear health or other conditions in addition to, or instead of, the ear wax buildup condition 146. For example, the one or more processors 106 can check for ear wax buildup, ear tip blockage, ear canal fluid, ear canal swelling, or any combination thereof, based on one or more audio propagation conditions determined based on the reflected audio signal 122.

According to an aspect, the device 102 is configured to send a signal to a user interface device to indicate that an ear health or other condition has been detected. For example, in some implementations the device 102 includes a modem 150 configured to communicate with a second device 152 via wireless transmission over a communication channel 154. To illustrate, the communication channel 154 may include or correspond to a wired connection between the device 102 and the second device 152, a wireless connection between the device 102 and the second device 152, or both. The device 102 can send an indication of a detected condition to the second device 152 for display to a user of the device 102. Alternatively, the device 102 may include one or more user interface devices, such as a display screen, a visual indicator (e.g., a light emitting diode indicator), an audio interface (e.g., voice output via the speaker 110), a haptic indicator, etc., which may be activated to notify the user that an ear health or other condition has been detected.

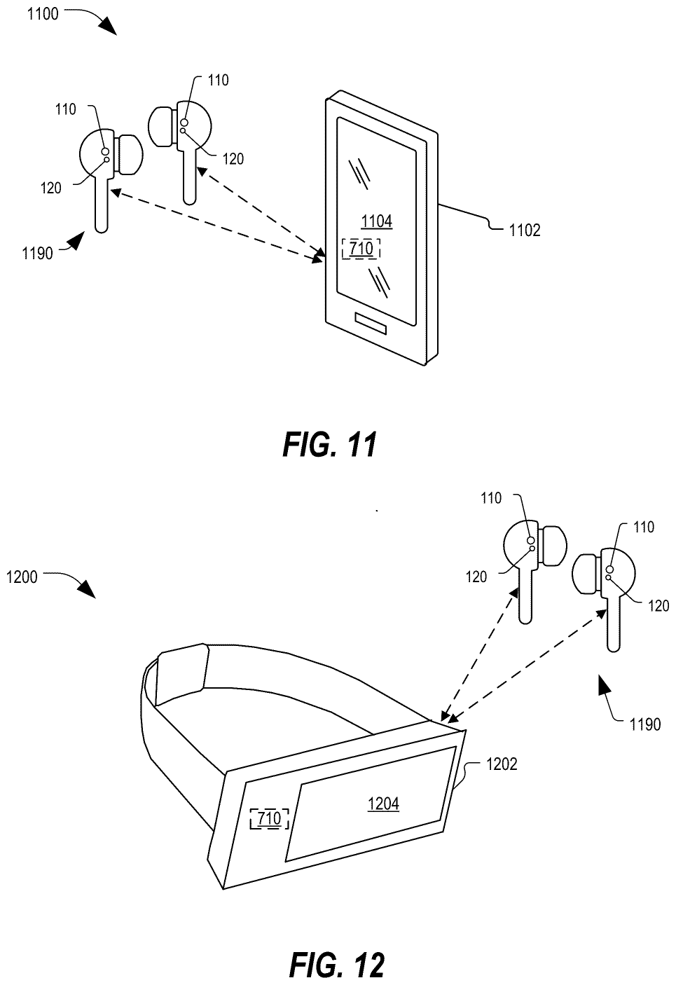

Although FIG. 1 illustrates that the device 102 includes the earphone 104 (e.g., the one or more processors 106 of the device 102 are integrated in the earphone 104 along with the speaker 110 and the feedback microphone 120), in other implementations the one or more processors 106 are not included in the earphone 104. For example, the device 102 including the one or more processors 106 can be implemented as a mobile phone, wearable device, or other device that is distinct from and coupled to the earphone 104, and the earphone 104 includes the speaker 110 and the feedback microphone 120, such as illustrated in FIGS. 11-14.

Although the operation of the device 102 is described in conjunction with determining the sound reflection characteristics 140, the change over time 144, and the ear wax buildup condition 146 for a single ear of the user, it should be understood that the above-described operations can be performed for each ear of the user. For example, in some implementations the earphone 104 corresponds to one earbud of a pair of earbuds, such as illustrated in FIG. 8. In such implementations, each earbud of the pair of earbuds includes a speaker 110, a feedback microphone 120, an active noise cancellation system 130, and a reflected signal analyzer 160 so that each earbud individually performs ear health condition monitoring for a respective single ear.

In other implementations in which the earphone 104 corresponds to one earbud of a pair of earbuds, each earbud can include a speaker 110, a feedback microphone 120, and an active noise cancellation system 130, but only a first earbud of the pair of earbuds includes the reflected signal analyzer 160. In such implementations, the second earbud can transmit reflection data, such as a representation of the received signal 124, the adaptive filter weights 132, or both, to the first earbud, and the first earbud can perform ear health condition monitoring and maintenance of the sound reflection characteristics history 142 for both ears.

In some implementations, two earphones 104 are integrated into a single device 102, such as a headset as illustrated in FIG. 9 or an extended reality headset as illustrated in FIG. 10. In such implementations, the reflected signal analyzer 160, the active noise cancellation system 130, or both, may be configured to perform signal processing and/or ear health condition monitoring for each of the user's ears. In implementations in which the reflected signal analyzer 160 is integrated in a separate device 102 that is communicatively coupled to one or more earphones 104, such as illustrated in FIGS. 11-14, the reflected signal analyzer 160 is configured to receive and process reflection data (e.g., a representation of the received signal 124, the adaptive filter weights 132, or both) corresponding to both of the user's ears, and in some cases can also perform ear health condition monitoring for several users concurrently.

The system 100 thus facilitates detection of an ear health condition during use of the earphones 104. Testing for the ear health condition can be performed during playback of user selected content and therefore can be performed during normal operation of the device 102 by the user and without the user's notice. In addition, because conventional earphone devices often include an ANC component that performs adaptive filtering for noise cancellation, the disclosed techniques can be implemented with reduced changes to existing designs as compared to fully customized hardware and/or software implementations. Because an ear health condition such as the ear wax buildup condition 146 may develop slowly without being noticed by the user, automatic testing performed by the device 102 can enable the user to become aware of the condition and to take action to remedy the condition, thus improving the user's ear health and also improving the user's listening experience.

FIG. 2 is a diagram of particular aspects of the system of FIG. 1, in accordance with some examples of the present disclosure. In particular, FIG. 2 highlights an example of components that can be implemented in the device 102, according to a particular implementation.

In the example illustrated in FIG. 2, the active noise cancellation system 130 includes a finite impulse response (FIR) filter 210 coupled to a subtracter 212. Conventionally, the active noise cancellation system 130 operates to generate an output signal 112 to be played out by the speaker 110 that at least partially cancels noise from external sources. To illustrate, the device 102 includes a reference microphone 230 that generates an external noise signal 232 that is based on external noise detected by the reference microphone 230 and that is provided to the active noise cancellation system 130. The active noise cancellation system 130 can use an FIR filter to generate an anti-noise signal based on the external noise signal 232 so that, when played out at the speaker 110, the anti-noise signal substantially cancels the external noise at the user's ear. During conventional active noise cancellation, the active noise cancellation system 130 samples the received signal 124 from the feedback microphone 120 (also referred to as an “error microphone”), which includes a combination of the external noise and the anti-noise signal, and dynamically adjusts adaptive filter weights to increase the effectiveness of the noise cancellation. The anti-noise signal can be combined with a playback audio signal 222 (e.g., the user-selected audio content 190) from a playback audio source 220, such as audio from a media player, a gaming engine, a voice call, etc., to form the output signal 112 that is provided to the speaker 110.

According to an aspect of the present disclosure, the active noise cancellation system 130 uses one or more sets of FIR filters and subtracters, illustrated as the representative FIR filter 210 and the representative subtracter 212, to determine reflection characteristics that can be associated with one or more ear health conditions. The FIR filter 210 processes the output signal 112 to generate an FIR output that substantially matches the received signal 124. The output of the FIR filter 210 is subtracted from the received signal 124 at the subtracter 212, and the active noise cancellation system 130 samples the resulting signal 214 and adjusts the adaptive filter weights 132 so that the resulting signal 214 is approximately zero.

The resulting adaptive filter weights 132 are indicative of an impulse response 280 that includes a first component 282 and a second component 286 representing a response at the feedback microphone 120 to a hypothetical impulse (e.g., a Dirac delta function) that is generated at a time T=0 from the speaker 110. The first component 282 includes a first peak 284 that corresponds to direct acoustic coupling between the speaker 110 and the feedback microphone 120, and the second component 286 includes a second peak 288 that corresponds to indirect acoustic coupling between the speaker 110 and the feedback microphone 120 via reflection from the eardrum 184. As illustrated by a time difference between the first peak 284 at a time T1 and the second peak 288 at a time T2, the reflection signal is time delayed due to the longer sound propagation path (also referred to as the “reflection path”) from the speaker 110 to the eardrum 184 and back to the feedback microphone 120. The direct coupling distance for the first peak 284 is generally on the order of millimeters (mm), while the reflection path for the second peak 288 is based on the length of the ear canal 186, which is approximately 2.5 centimeters (cm) for most people, resulting in approximately a 5 cm total reflection path length. The second peak 288 is also lower than the first peak 284, indicating lower relative signal strength or intensity, due to attenuation along the reflection path.

The extent of the attenuation along the reflection path can be affected by one or more conditions such as ear wax buildup in the ear canal 186. To illustrate, if the amount of audio reflection is reduced due to obstruction caused by ear wax buildup, the intensity (height) of the second peak 288 will also reduce.

Operation of the FIR filter 210 to generate an output signal that cancels the received signal 124 (e.g., that substantially matches the impulse response 280) results in values of the adaptive filter weights 132 that, when graphed as a function of tap position (also referred to as “weight index”), can be observed to generally match the shape of the impulse response 280 including the relative delays and the relative magnitudes associated with the peaks 284, 288, as described further with reference to FIGS. 3-5. The values and the changes over time of the adaptive filter weights 132 can therefore be used to detect changes in the reflection path of the ear canal 186 that can indicate one or more ear health conditions.

In some implementations, the processing described above to generate the adaptive filter weights 132 corresponding to the impulse response 280 is performed for each frequency bin that is used by the active noise cancellation system 130 for conventional active noise cancellation. In an example in which the device 102 uses a fast Fourier transform (FFT) of length 512 to transform an audio signal into 257 frequency bins that are processed for conventional active noise cancellation, for an FIR filter in the active noise cancellation system 130 running at 16 kilohertz (kHz) (e.g., a wide-band signal), 8 kHz is the highest frequency component contained in the signal, and the 8 kHz range is divided into 257 frequency bins. In this example, the active noise cancellation system 130 can include a set of 257 FIR filters for anti-noise processing.

In addition to the set of FIR filters that are used for anti-noise processing, another set of one or more FIR filters is used to determine the sound reflection characteristics 140 of FIG. 1. To illustrate, a duplicate of the FIR filter 210 can be run in each of the 257 frequency bins to determine the reflection characteristics for each frequency bin, such as by generating, for each frequency bin, a set of the adaptive filter weights 132 that represent the impulse response for that frequency bin. In other implementations, improved efficiency in determining the sound reflection characteristics 140 can be attained by limiting adaptive filter processing to one or more frequency bins in a lower frequency range, such as in the range from 50 Hz to 250 Hz, where the reflections are strongest. To illustrate, higher-frequency sounds are more easily absorbed by human flesh than lower-frequency sounds; as a result, lower-frequency reflections are less attenuated than high-frequency sounds along the reflection path. In a particular example, reflection signal filtering performed for a single frequency bin at 150 Hz (e.g., a frequency bin that corresponds to a frequency range that includes 150 Hz) can provide sufficient information regarding conditions in the ear canal 186 to detect various ear health conditions including the ear wax buildup condition 146.

According to an aspect, the device 102 is configured to perform a calibration operation 260. During the calibration operation 260, the device 102 is configured to initiate playback of a calibration signal 262 into the ear canal 186 by providing the calibration signal 262 as the output signal 112 for playout at the speaker 110. For example, the calibration signal 262 can include one or more tones in the frequency range of 50-250 hertz (Hz), such as at 150 Hz. The calibration operation 260 may also include determining the impulse response 280 of the ear canal, where the impulse response 280 is associated with the calibration signal 262. The reflected signal analyzer 160 can determine a baseline sound reflection characteristic 264 based on a height of a peak associated with the impulse response 280 and corresponding to reflection from the eardrum 184. The baseline sound reflection characteristic 264, data such as one or more thresholds generated based on the baseline sound reflection characteristic 264, or a combination thereof, can be saved in the sound reflection characteristics history 142.

After calibration, the device 102 can periodically or occasionally perform an ear health determination operation to determine whether one or more audio propagation conditions 250 are detected. For example, the device 102 can perform a reflection signal analysis once a day, once every two days, once a week, or according to some other time interval, during normal use of the device 102 (e.g., during playback of a user-selected playback audio signal 222 from the playback audio source 220).

To illustrate, the active noise cancellation system 130 processes the received signal 124 during playback of the user-selected playback audio signal 222 to determine the adaptive filter weights 132 associated with the received signal 124, and the reflected signal analyzer 160 determines whether one or more audio propagation conditions 250 are detected. The one or more audio propagation conditions 250 can be detected at least partially based on comparison to the baseline sound reflection characteristic 264 to identify changes in the sound reflection characteristics of the ear canal 186 over time. The one or more audio propagation conditions 250 include the ear wax buildup condition 146, an ear tip blockage condition 252, an ear canal fluid condition 254, and an ear canal swelling condition 256.

The ear wax buildup condition 146 can be detected based on a reduction in a height of a peak of the adaptive filter weights 132 that is associated with a reduced height of the second peak 288, as described further with reference to FIG. 3 and FIG. 4.

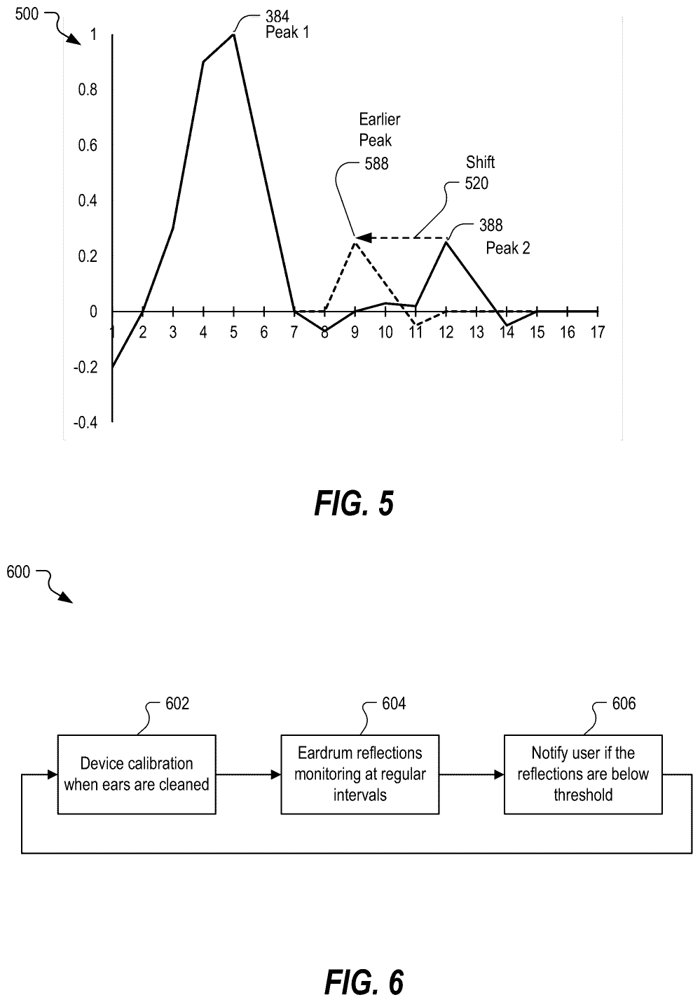

The ear tip blockage condition 252 results from a blockage (or partial blockage) in the aperture of the ear tip of an in-ear style earphone. In particular, an “ear tip” is typically a removable portion of an in-ear style earphone that is adapted to form a seal between the earphone and the user's ear canal and that has a channel that allows sound to travel from the speaker 110 into the ear canal. Ear tips are typically made of silicon or foam and include a filter just outside of the speaker 110 that can be blocked by dirt, dust, or ear wax. When the ear tip filter, or any other portion of the channel through the ear tip, is blocked, sound played out by the device 102 will be perceived by the user as muffled, with high-frequency content more significantly attenuated than low-frequency content. The ear tip blockage condition 252 causes sound from the speaker 110 to be reflected by the obstruction in the ear tip, so that the dominant sound reflection path from the speaker 110 to the feedback microphone 120 is much shorter than the refection path for sound reflected from the eardrum 184. The shorter reflection path results in a reflection peak occurring earlier (e.g., between time T1 and T2) in the impulse response 280 than for reflections from the eardrum 184. An example of detection of an earlier reflection peak is described in further detail with reference to FIG. 5.

The ear canal fluid condition 254 results from the presence of water or other fluid in the ear canal 186. In general, sound reflections from water have higher intensity than sound reflections from human flesh or ear wax. As a result, in response to the height of the second peak 288 being larger than was observed during calibration, the reflected signal analyzer 160 can determine that the reflection is not due to ear wax and instead is due to water or some other obstruction in the ear canal 186.

The ear canal swelling condition 256 results from the generally cylindrical-shaped space inside the ear canal 186 becoming smaller, such as due to swelling from infection or injury. The reduced space in the ear canal 186 causes the second peak 288 to have reduced height as compared to baseline. Because swelling tends to occur over a shorter time period than ear wax buildup, such as in 1-2 days as compared to weeks or months for ear wax buildup, the reduction of the height of the second peak 288 due to swelling occurs much more rapidly than for ear wax buildup. This difference can be used by the reflected signal analyzer 160 to distinguish between the ear wax buildup condition 146 and the ear canal swelling condition 256.

In response to detecting one or more of the audio propagation conditions 250, the device 102 is configured to send a signal 292 to a user interface device to indicate which audio propagation condition(s) 250 have been detected. In the example illustrated in FIG. 2, the device 102 includes, or is coupled to, a display device 290 that is configured to receive the signal 292 and to display an indication of the one or more audio propagation conditions 250 that have been detected. In some implementations, the signal 292 also conveys information regarding remedial action that the user can take. For example, for the ear wax buildup condition 146, the signal 292 may cause the display device 290 to display a recommendation that the user perform ear cleaning. As another example, for the ear tip blockage condition 252, the signal 292 may cause the display device 290 to display a recommendation to remove and clean the ear tips or to replace the ear tips to improve the sound quality for the user. Other examples include recommending that the user seek medical attention,

Although the sound reflection processing at the active noise cancellation system 130 is described in the context of using FIR filters that may already be present in a conventional ANC system, in other implementations FIR filtering can be performed through software executing at the one or more processors 106 (e.g., a DSP) of the device 102.

Although in the implementation depicted in FIG. 2 the reflected signal analyzer 160 is configured to detect four audio propagation conditions 250, in other implementations the reflected signal analyzer 160 may be configured to omit testing for one or more of the audio propagation conditions 250, to include testing for one or more other audio propagation conditions in addition to, or in place of, the illustrated audio propagation conditions 250, or a combination thereof.

FIG. 3 is a diagram of particular aspects of the system of FIG. 1, in accordance with some examples of the present disclosure. In particular, FIG. 3 highlights values of the adaptive filter weights 132 corresponding to the impulse response 280 in the absence of any audio propagation condition(s) 250, such as the impulse response 280 resulting from the calibration operation 260 following an ear cleaning, according to a particular implementation.

In the example illustrated in FIG. 3, a graph 300 depicts a curve corresponding to normalized values of each filter weight of a set of the adaptive filter weights 132, where the filter weight indexes are represented as numbers along the horizontal axis and the corresponding filter weight values are represented as the height of the curve at each filter weight index. Although illustrated as a curve to demonstrate correspondence to the impulse response 280, it should be understood that the adaptive filter weights 132 are generated as a set of discrete weight values.

In the illustrated example, there are seventeen filter weights, with indices from 1 to 17. Each filter weight is associated with a corresponding tap of a filter, such as the FIR filter 210, and an amount of delay associated with each filter weight is proportional to the index of the filter weight. For example, filter weight 2 has a value of 0 and corresponds to two units of delay, filter weight 3 has a value of 0.3 and corresponds to three units of delay, filter weight 4 has a value of 0.9 and corresponds to four units of delay, etc.

As illustrated, the curve has a first peak 384 corresponding to the first peak 284 of the impulse response 280, indicating a direct path between the speaker 110 and the feedback microphone 120. The curve also has a second peak 388 corresponding to the second peak 288 of the impulse response 280, indicating a reflection path between the speaker 110 and the feedback microphone 120. A threshold 330 is illustrated as a horizontal line corresponding to a value of approximately 0.1.

The threshold 330 may be determined by the device 102 (e.g., set by processor(s) 106 during the calibration operation 260) based on the height 320 of the second peak 388. As illustrated, the height 320 of the second peak 388 is approximately 0.25. The processor(s) 106 can set the threshold 330 to a value representing a 60% reduction in the height 320 of the second peak 388, such as using the expression:

threshold=(peak value)−(60% of peak value)=0.25−(0.6*0.25)=0.1.

In other implementations, the threshold 330 can be determined based on one or more other percentages of the height 320 or based on one or more other techniques. For example, in some implementations, the threshold 330 can be determined based on a percentage of the overall peak-to-valley height associated with the second peak 388 rather than the amplitude of the curve at the second peak 388. To illustrate, the second peak 388 is between local minima of approximately −0.05 at weight 8 and weight 14 and therefore has a peak-to-valley height of approximately 0.3, and the threshold 330 at 0.1 can be determined based on the expression:

threshold=(peak value)−(50% of peak-to-valley height).

According to some aspects, the reflected signal analyzer 160 is configured to determine the condition 146 corresponding to ear wax buildup based on the height 320 of the second peak 388 falling below the threshold 330, as illustrated in FIG. 4. The threshold 330, the set of adaptive filter weights 132, one or more other parameters, or any combination thereof, can be stored in the memory 108, such as in the sound reflection characteristics history 142 and corresponding to the baseline sound reflection characteristic 264.

FIG. 4 is a diagram of particular aspects of the system of FIG. 1, in accordance with some examples of the present disclosure. In particular, FIG. 4 highlights values of the adaptive filter weights 132 corresponding to the impulse response 280 after buildup of ear wax, according to a particular implementation.

In the example illustrated in FIG. 4, a graph 400 depicts a curve corresponding to values of each filter weight of a set of the adaptive filter weights 132, showing that the second peak 388 occurs at weight 13 as compared to weight 12 in the graph 300, and that the height 320 of the second peak 388 has fallen below the threshold 330.

According to some aspects, detection of the height 320 falling below the threshold 330 causes the reflected signal analyzer 160 to determine that the ear wax buildup condition 146 has occurred. According to other aspects, in response to detecting the height 320 falling below the threshold 330, the reflected signal analyzer 160 determines how quickly the second peak 288 has fallen from its baseline value to distinguish between the ear wax buildup condition 146 and the ear canal swelling condition 256. For example, the reflected signal analyzer 160 may determine a rate of change of the height 320 of the second peak 388 at least partially based on the sound reflection characteristics history 142, and may select the ear wax buildup condition 146 based on a relatively slow rate of change or select the ear canal swelling condition 256 based on a relatively fast rate of change.

In some implementations, the rate of change of the height 320 is determined based on determining an amount of time that has elapsed between the measurement of the baseline sound reflection characteristic 264 (e.g., as illustrated in the graph 300) and detecting that the height 320 has fallen below the threshold 330 (e.g., as illustrated in the graph 400). In other implementations, the rate of change of the height 320 is primarily determined based on one or more time periods immediately preceding the height 320 falling below the threshold 330. In such implementations, the reflected signal analyzer 160 can calculate changes in the height 320 between each of the most recent ear health measurements in the sound reflection characteristics history 142 and compare the calculated changes to a rate of change threshold to determine whether the height 320 falling below the threshold 330 is the result of a gradual decrease (indicative of ear wax buildup) or a sudden decrease (indicative of ear canal swelling). Thus, in an example where a user experiences relatively slow ear wax accumulation (e.g., a relatively small decrease of the height 320 over time) after calibration, followed by an ear infection several months after calibration that causes the height 320 to quickly drop below the threshold 330, the reflected signal analyzer 160 can determine that the immediate cause of the reduced height 320 is ear canal swelling instead of ear wax buildup based on the rate of change between the recent measurements being greater than the rate of change threshold.

FIG. 5 is a diagram of particular aspects of the system of FIG. 1, in accordance with some examples of the present disclosure. In particular, FIG. 5 highlights values of the adaptive filter weights 132 corresponding to the impulse response 280 after an ear tip of the earphone 104 has become blocked, according to a particular implementation.

In the example illustrated in FIG. 5, a graph 500 depicts a curve corresponding to values of each filter weight of a set of the adaptive filter weights 132, showing an earlier peak 588 (illustrated with dashed lines) that corresponds to reflection from the ear tip blockage, which occurs at a lower filter weight index (filter weight 9) than the second peak 388 of FIG. 3 (illustrated with solid lines). The lower filter weight index of the earlier peak 588 is due to the shorter reflection path caused by reflection from the ear tip blockage as compared to reflection from the eardrum 184. Because the ear tip blockage causes the majority of the output audio signal 114 to be reflected at the ear tip instead of at the eardrum 184, the second peak 388 will be significantly attenuated, or undetectable, as compared to the earlier peak 588. Ear tip blockage can therefore generate the appearance that a shift 520 has been applied to the second peak 388 of FIG. 3 to move the second peak 388 to the left from its baseline position.

According to some aspects, the reflected signal analyzer 160 is configured to detect the shift 520 and compare the shift 520 to a shift threshold to determine the ear tip blockage condition 252. In an example, the reflected signal analyzer 160 determines the shift 520 by comparing the position of the earlier peak 588 (at filter weight 9) to the position of the second peak 388 (at filter weight 12) in the baseline sound reflection characteristic 264. According to some aspects, detection of the shift 520 exceeding the shift threshold causes the reflected signal analyzer 160 to determine that the ear tip blockage condition 252 has occurred.

Although FIGS. 3-5 depict implementations in which the adaptive filter weights 132 include 17 weights and analysis is performed using normalized values of the filter weights, it should be understood that in other implementations, fewer than 17 filter weights or more than 17 filter weights may be used, and analysis may be performed using normalized weight values, unnormalized weight values, or any other representation of the weight values determined by the active noise cancellation system 130.

FIG. 6 is a diagram of particular aspects of the system of FIG. 1, in accordance with some examples of the present disclosure. In particular, FIG. 6 highlights an example of operations 600 that may be performed by the device 102, according to a particular implementation.

In the example illustrated in FIG. 6, the operations 600 include device calibration when the user's ears have been cleaned, at block 602. For example, the device 102 may prompt the user to perform an ear cleaning. The user can indicate, via a user interface, that an ear cleaning has been performed, in response to which the device 102 conducts the calibration operation 260. For example, the device 102 causes the calibration signal 262 to be played out at the speaker 110, and the resulting adaptive filter weights 132 can be included in the baseline sound reflection characteristic 264. In addition, or alternatively, the device 102 can determine one or more other parameters, such as a timestamp of the calibration operation 260, the height and position of the second peak 388, one or more threshold values such as the threshold 330, etc., to be included in the baseline sound reflection characteristic 264. The baseline sound reflection characteristics 264 can be stored in sound reflection characteristics history 142 in the memory 108.

According to some implementations, the device 102 can support multiple users, such as when the reflected signal analyzer 160 is integrated in an earphone 104 that is shared by multiple users, or when multiple users each use a separate set of earphones that are coupled to the device 102 (e.g., multiple passengers using earphones coupled to a vehicle entertainment system). In such implementations, the device 102 may be configured to distinguish between the users (e.g., via user login or biometric data corresponding to user profiles) and can perform the operations 600 and maintain separate sound reflection characteristics for each user. In some implementations, the device 102 can distinguish between users based on differences between the baseline sound reflection characteristic 264 or other ear measurement data that may be generated by the device 102.

The operations 600 also include eardrum reflections monitoring at regular intervals, at block 604. In some examples, the eardrum reflections monitoring is performed during playback of the user-selected audio content 190. For example, the FIR filter 210 can process the output signal 112 including the user-selected playback audio signal 222 from the playback audio source 220, such as by processing one or more frequency bands in the 50-250 Hz range, to generate the adaptive filter weights 132 that cause the resulting signal 214 to be as close to zero as possible in the one or more frequency bands. In other examples, the eardrum reflections monitoring is performed using the calibration signal 262, instead of during playback of the user-selected audio content 190, to generate the adaptive filter weights 132. The reflected signal analyzer 160 processes the resulting adaptive filter weights 132 to determine the change over time 144 of the sound reflection characteristics 140 for use in determining an ear health condition.

According to an aspect, the device 102 attempts to perform the eardrum reflections monitoring according to a schedule, such as every day or every other day. If the user does not use the device 102 to listen to audio content via the earphone 104 for more than a threshold number of days, the device 102 can prompt the user to put on the earphone 104 to perform a monitoring operation using the calibration signal 262 or any other suitable audio content that is available for playback at the device 102.

The operations 600 include notifying the user if the reflections are below a threshold, at block 606. For example, when the height 320 of the second peak 388 has fallen below the threshold 330, the device 102 notifies the user of the ear wax buildup condition 146 (or, in some implementations, the ear canal swelling condition 256, as described above). The device 102 can notify the user via a user interface component at the second device 152, via the display device 290, or via one or more other user interface components of the device 102 and/or the earphone 104 such as an audio message played out at the speaker 110, a visual indicator, haptic feedback, etc.

According to some implementations, the operations 600 also include notifying the user of an ear canal obstruction condition, such as the ear canal fluid condition 254, if the height of the second peak 388 is larger than a baseline height by a threshold amount. In some implementations, the operations 600 include notifying the user of the ear tip blockage condition 252 if the shift 520 exceeding a shift threshold is detected.

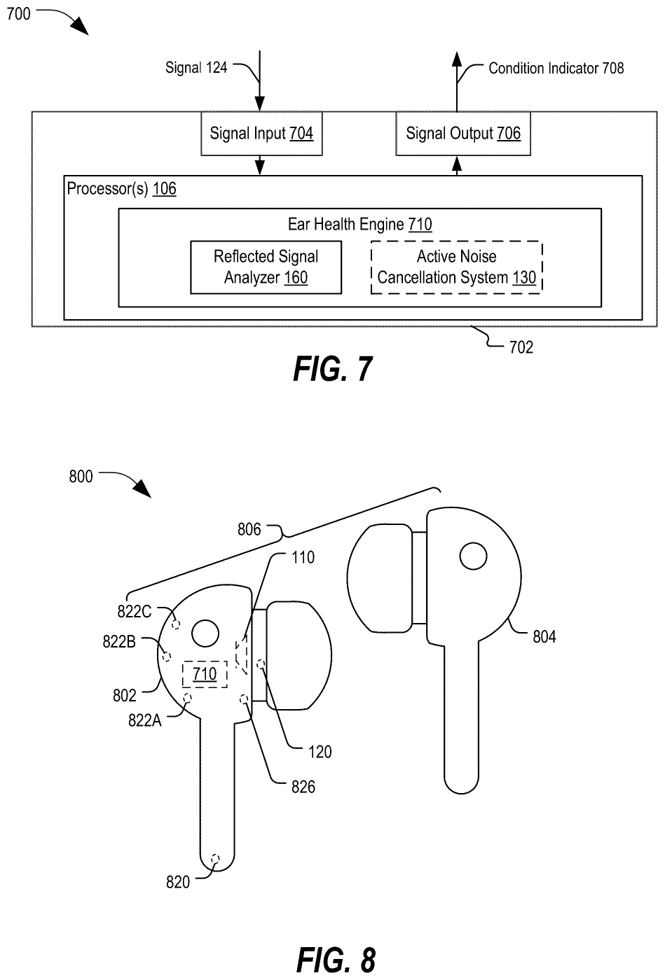

FIG. 7 depicts an implementation 700 of the device 102 as an integrated circuit 702 that includes the one or more processors 106. The processor(s) 106 include one or more components of an ear health engine 710, including the reflected signal analyzer 160 and optionally including the active noise cancellation system 130. The integrated circuit 702 also includes signal input circuitry 704, such as one or more bus interfaces, to enable the signal 124 (or, optionally, the adaptive filter weights 132) to be received for processing. The integrated circuit 702 also includes signal output circuitry 706, such as a bus interface, to enable sending of a condition indicator 708 from the integrated circuit 702. For example, the condition indicator 708 may include a signal to one or more user interface components or remote devices, such as the signal 292 for the display device 290, to generate an output in response to detecting an ear health condition, such as one or more of the audio propagation conditions 250.

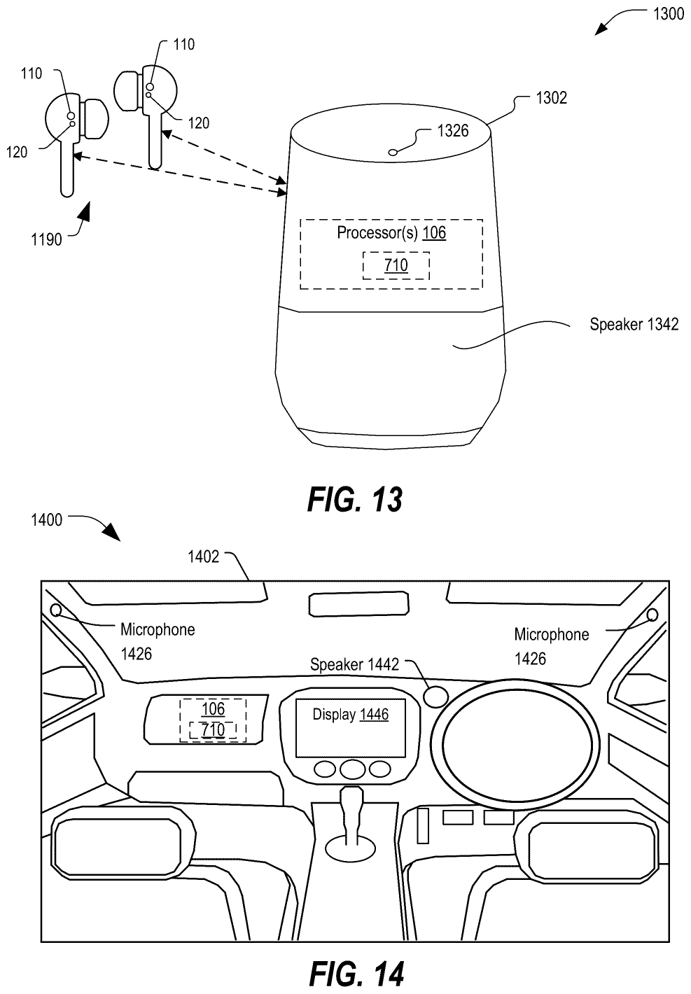

The integrated circuit 702 enables implementation of ear health condition monitoring as a component in a system that includes a feedback microphone, such as a pair of earbuds as depicted in FIG. 8, a headset as depicted in FIG. 9, or an extended reality (e.g., a virtual reality, mixed reality, or augmented reality) headset as depicted in FIG. 10. The integrated circuit 702 also enables implementation of ear health condition monitoring as a component in a system that receives a feedback microphone signal from an earphone, such as a mobile phone or tablet as depicted in FIG. 11, a wearable electronic device as depicted in FIG. 12, a voice assistant device as depicted in FIG. 13, or a vehicle as depicted in FIG. 14.

FIG. 8 depicts an implementation 800 of the device 102 in which the earphone 104 corresponds to an in-ear style earphone, illustrated as a pair of earbuds 806 including a first earbud 802 and a second earbud 804. Although earbuds are depicted, it should be understood that the present technology can be applied to other in-ear, on-ear, or over-ear playback devices. Various components, such as the ear health engine 710, are illustrated using dashed lines to indicate internal components that are not generally visible to a user.

The first earbud 802 includes the ear health engine 710, the speaker 110, a first microphone 820, such as a high signal-to-noise microphone positioned to capture the voice of a wearer of the first earbud 802, an array of one or more other microphones configured to detect ambient sounds and that may be spatially distributed to support beamforming, illustrated as microphones 822A, 822B, and 822C, the feedback microphone 120 proximate to the wearer's ear canal, and a self-speech microphone 826, such as a bone conduction microphone configured to convert sound vibrations of the wearer's ear bone or skull into an audio signal. In a particular implementation, the microphones 822A, 822B, and 822C correspond to multiple instances of the reference microphone 230, and audio signals generated by the microphones 820 and 822A, 822B, and 822C are used to generate the external noise signal 232.

The ear health engine 710 is coupled to the speaker 110 and the feedback microphone 120 and is configured to perform ear health condition monitoring as described above. According to some aspects, in response to determining an ear health condition associated with a first ear of the wearer, such as the ear wax buildup condition 146, the first earbud 802 causes an audible notification, such as a voice message, to be played out via the speaker 110 to inform the wearer of the ear health condition. The second earbud 804 can be configured in a substantially similar manner as the first earbud 802 and operable to detect an ear health condition associated with the second ear of the wearer in conjunction with, or independently of, operation of the first earbud 802.

In some implementations, the earbuds 802, 804 are configured to automatically switch between various operating modes, such as a passthrough mode in which ambient sound is played via the speaker 110, a playback mode in which non-ambient sound (e.g., streaming audio corresponding to a phone conversation, media playback, a video game, etc.) is played back through the speaker 110, and an audio zoom mode or beamforming mode in which one or more ambient sounds are emphasized and/or other ambient sounds are suppressed for playback at the speaker 110. In other implementations, the earbuds 802, 804 may support fewer modes or may support one or more other modes in place of, or in addition to, the described modes.

In an illustrative example, the earbuds 802, 804 can automatically transition from the playback mode to the passthrough mode in response to detecting the wearer's voice, and may automatically transition back to the playback mode after the wearer has ceased speaking. In some examples, the earbuds 802, 804 can operate in two or more of the modes concurrently, such as by performing audio zoom on a particular ambient sound (e.g., a dog barking) and playing out the audio zoomed sound superimposed on the sound being played out while the wearer is listening to music (which can be reduced in volume while the audio zoomed sound is being played). In this example, the wearer can be alerted to the ambient sound associated with the audio event without halting playback of the music. Ear health monitoring can be performed by the ear health engine 710 in any of the modes. For example, the audio played out at the speaker 110 during the passthrough mode can also be used as the playback audio signal 222 for performing ear health monitoring.

FIG. 9 depicts an implementation 900 in which the device 102 is a headset device 902. The headset device 902 includes a speaker 110 and a feedback microphone 120 in each earcup, and the ear health engine 710 is integrated in the headset device 902 and configured to perform ear health monitoring for each of the user's ears as described above. The headset device 902 may be configured to provide an audible notification to a wearer of the headset device 902 to notify the wearer of a detected ear health condition, such as based on determining the ear wax buildup condition 146, the ear tip blockage condition 252 (e.g., when the headset device 902 includes in-ear type earphones), the ear canal fluid condition 254, the ear canal swelling condition 256, or a combination thereof.

FIG. 10 depicts an implementation 1000 in which the device 102 includes a portable electronic device that corresponds to an extended reality (e.g., a virtual reality, mixed reality, or augmented reality) headset 1002. The headset 1002 includes a visual interface device and earphone devices, illustrated as over-ear earphone cups that each include a speaker 110 and a feedback microphone 120. The visual interface device is positioned in front of the user's eyes to enable display of augmented reality, mixed reality, or virtual reality images or scenes to the user while the headset 1002 is worn.

The ear health engine 710 is integrated in the headset 1002 and configured to perform ear health monitoring for each of the user's ears as described above. The headset 1002 may be configured to provide an audible notification, a visual notification, or both, to a wearer of the headset 1002 to notify the wearer of a detected ear health condition, such as based on determining the ear wax buildup condition 146, the ear tip blockage condition 252 (e.g., when the headset 1002 includes in-ear type earphones), the ear canal fluid condition 254, the ear canal swelling condition 256, or a combination thereof.

FIG. 11 depicts an implementation 1100 in which the device 102 includes a mobile device 1102, such as a phone or tablet, coupled to earphones 1190, such as a pair of earbuds, as illustrative, non-limiting examples. The ear health engine 710 is integrated in the mobile device 1102 and configured to perform ear health monitoring for each of a user's ears as described above.

Each of the earphones 1190 includes at least one speaker 110 and feedback microphone 120. Each earphone 1190 is configured to wirelessly receive audio data from the mobile device 1102 for playout at the speaker 110, such as the playback audio signal 222 or the calibration signal 262.

In some implementations, each of the earphones 1190 includes the active noise cancellation system 130 that includes the FIR filter 210 and subtracter 212 and that is configured to generate the adaptive filter weights 132. In such implementations, the earphones 1190 are each configured to transmit its respective set of adaptive filter weights 132 to the mobile device 1102 for processing at the ear health engine 710.

In other implementations, the earphones 1190 do not generate the adaptive filter weights 132, and instead the earphones 1190 transmit the received signal 124 from each of the earphones 1190 to the mobile device 1102. In such implementations, the ear health engine 710 at the mobile device 1102 is configured to process the received signals 124 to generate the adaptive filter weights 132.

The mobile device 1102 may be configured to provide an audible notification (such as via an audio signal transmitted to the earphones 1190 for playout), a visual notification provided via a display screen 1104, or both, to a user of the mobile device 1102 to notify the user of a detected ear health condition, such as based on determining the ear wax buildup condition 146, the ear tip blockage condition 252, the ear canal fluid condition 254, the ear canal swelling condition 256, or a combination thereof.

FIG. 12 depicts an implementation 1200 in which the device 102 includes a wearable electronic device 1202, illustrated as a “smart watch,” coupled to the earphones 1190, such as a pair of earbuds, as illustrative, non-limiting examples. The ear health engine 710 is integrated in the wearable electronic device 1202 and configured to perform ear health monitoring for each of a user's ears as described above.

Each of the earphones 1190 includes at least one speaker 110 and feedback microphone 120. Each earphone 1190 is configured to wirelessly receive audio data from the wearable electronic device 1202 for playout at the speaker 110, such as the playback audio signal 222 or the calibration signal 262. Each earphone 1190 is also configured to wirelessly transmit information regarding audio data captured by the feedback microphones 120 to the wearable electronic device 1202.

In some implementations, each of the earphones 1190 includes the active noise cancellation system 130 that includes the FIR filter 210 and subtracter 212 and that is configured to generate the adaptive filter weights 132, as described previously. In such implementations, the earphones 1190 are configured to transmit the adaptive filter weights 132 from each of the earphones 1190 to the wearable electronic device 1202 for processing at the ear health engine 710.

In other implementations, the earphones 1190 do not generate the adaptive filter weights 132, and instead the earphones 1190 transmit the received signal 124 from each of the earphones 1190 to the wearable electronic device 1202. In such implementations, the ear health engine 710 at the wearable electronic device 1202 is configured to process the received signals 124 to generate the adaptive filter weights 132.

The wearable electronic device 1202 may be configured to provide an audible notification (via an audio signal transmitted to the earphones 1190 for playout), a visual notification provided via a display screen 1204, a notification via haptic feedback to the user, or any combination thereof, to notify the user of a detected ear health condition such as the ear wax buildup condition 146, the ear tip blockage condition 252, the ear canal fluid condition 254, the ear canal swelling condition 256, or a combination thereof.

FIG. 13 is an implementation 1300 in which the device 102 includes a wireless speaker and voice activated device 1302 coupled to the earphones 1190. The wireless speaker and voice activated device 1302 can have wireless network connectivity and is configured to execute an assistant operation, such as adjusting a temperature, playing music, turning on lights, etc. For example, assistant operations can be performed responsive to receiving a command after a keyword or key phrase (e.g., “hello assistant”).

The one or more processors 106 including the ear health engine 710 are integrated in the wireless speaker and voice activated device 1302 and configured to perform ear health monitoring for each of a user's ears as described above. The wireless speaker and voice activated device 1302 also includes a microphone 1326 and a speaker 1342 that can be used to support voice assistant sessions with users that are not wearing earphones.

Each of the earphones 1190 includes at least one speaker 110 and feedback microphone 120. Each earphone 1190 is configured to wirelessly receive audio data from the wireless speaker and voice activated device 1302 for playout at the speaker 110, such as the playback audio signal 222 or the calibration signal 262. Each earphone 1190 is also configured to wirelessly transmit information regarding audio data captured by the feedback microphones 120 to the wireless speaker and voice activated device 1302.

In some implementations, each of the earphones 1190 includes the active noise cancellation system 130 that includes the FIR filter 210 and subtracter 212 and that is configured to generate the adaptive filter weights 132, as described previously. In such implementations, the earphones 1190 are configured to transmit the adaptive filter weights 132 from each of the earphones 1190 to the wireless speaker and voice activated device 1302 for processing at the ear health engine 710.

In other implementations, the earphones 1190 do not generate the adaptive filter weights 132. In such implementations, the earphones 1190 transmit the received signal 124 from each of the earphones 1190 to the wireless speaker and voice activated device 1302, and the ear health engine 710 at the wireless speaker and voice activated device 1302 is configured to process the received signals 124 to generate the adaptive filter weights 132.

The wireless speaker and voice activated device 1302 may be configured to provide an audible notification (via an audio signal transmitted to the earphones 1190 for playout), a visual notification provided via a display screen, or both, to notify the user of a detected ear health condition, such as based on determining the ear wax buildup condition 146, the ear tip blockage condition 252, the ear canal fluid condition 254, the ear canal swelling condition 256, or a combination thereof.

FIG. 14 depicts an implementation 1400 in which the device 102 includes a vehicle 1402, illustrated as a car. The one or more processors 106 including the ear health engine 710 are integrated in the vehicle 1402 and configured to perform ear health monitoring for one or more occupants (e.g., passenger(s) and/or operator(s)) of the vehicle 1402 that are wearing earphones, such as the earphones 1190 (not shown). For example, the vehicle 1402 is configured to support multiple independent wireless or wired audio sessions with multiple occupants that are each wearing earphones, such as by enabling each of the occupants to independently stream audio, engage in a voice call or voice assistant session, etc., via their respective earphones, during which ear health monitoring may be performed. The vehicle 1402 also includes multiple microphones 1426, one or more speakers 1442, and a display 1446. The microphones 1426 and the speakers 1442 can be used to support, for example, voice calls, voice assistant sessions, in-vehicle entertainment, etc., with users that are not wearing earphones.

In some examples, the vehicle 1402 is configured to identify one or more occupants, such as via facial recognition, voice signature recognition, user weight, or other biometric recognition techniques; verbal self-identification; user login; one or more other occupant identification techniques, or any combination thereof. The one or more processors 106 can maintain user profiles including ear health data (e.g., the sound reflection characteristics history 142) for multiple occupants and, in response to determining that an identified occupant is participating in an audio session using earphones, can perform an ear health monitoring operation for that occupant.

The vehicle 1402 may be configured to provide an audible notification via an audio signal transmitted to an occupant's earphones for playout, a visual notification provided via the display 1446, or both, to notify the occupant of a detected ear health condition, such as based on determining the ear wax buildup condition 146, the ear tip blockage condition 252, the ear canal fluid condition 254, the ear canal swelling condition 256, or a combination thereof.

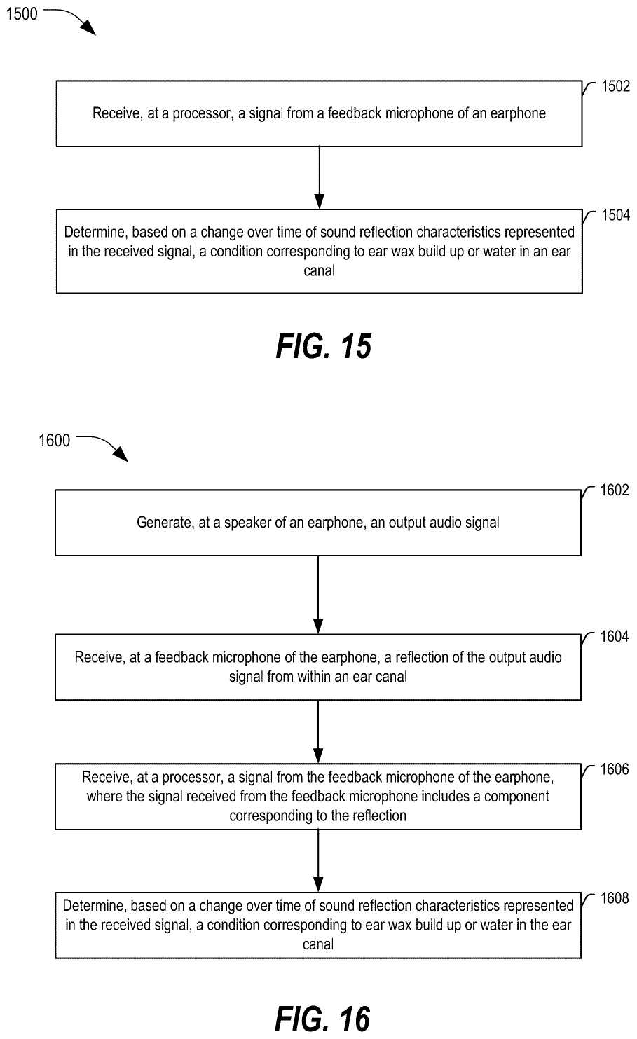

FIG. 15 depicts a particular implementation of a method 1500 of ear health condition detection. In a particular aspect, one or more operations of the method 1500 are performed by at least one of the active noise cancellation system 130, the reflected signal analyzer 160, the one or more processors 106, the device 102, the system 100, or a combination thereof.

The method 1500 includes, at block 1502, receiving, at a processor, a signal from a feedback microphone of an earphone. For example, the one or more processors 106 receive the signal 124 from the feedback microphone 120.

The method 1500 includes, at block 1504, determining, based on a change over time of sound reflection characteristics represented in the received signal, a condition corresponding to ear wax buildup or water in an ear canal. For example, the reflected signal analyzer 160 determines the ear wax buildup condition 146 or the ear canal fluid condition 254 based on the change over time 144 of the sound reflection characteristics 140.

According to some aspects, the sound reflection characteristics are determined based on adaptive filter weights of an active noise cancellation system. To illustrate, the sound reflection characteristics 140 can be based on the adaptive filter weights 132 of the FIR filter 210 generated by the active noise cancellation system 130. In some implementations, the sound reflection characteristics correspond to a height of a peak that is associated with the adaptive filter weights and that corresponds to a reflection from an eardrum, such as the height 320 of the second peak 388 as described for FIG. 3 and FIG. 4. In some such implementations, determining the condition corresponding to ear wax buildup is based on the height of the peak falling below a threshold, such as by comparing the height 320 to the threshold 330. In some such implementations, determining the condition corresponding to water in the ear canal is based on the height of the peak exceeding a calibration value, such as by determining the height of the second peak 388 is larger than a baseline height by a threshold amount.

In some implementations, the sound reflection characteristics are determined during playback of user-selected audio content at the earphone, such as when the user-selected audio content 190 is provided as the playback audio signal 222, and the FIR filter 210 processes one or more frequency bands of the received signal 124 to generate the adaptive filter weights 132 based on reflection of the playback audio signal 222 from the eardrum 184.

According to some aspects, the method 1500 includes performing a calibration operation. For example, the device 102 can perform the calibration operation 260 after the user has had an ear cleaning to generate the baseline sound reflection characteristic 264.

One benefit of performing ear health condition detection to identify a condition corresponding to ear wax buildup is that ear wax can build up gradually, and the corresponding effect of the ear wax buildup on the user's hearing may occur so slowly that the change is not noticed or is not perceivable by the user. By identifying the ear wax buildup condition, the user can be notified to take remedial action, resulting in improved hearing, better ear health, and an enhanced user experience. Another benefit of determining the ear wax buildup condition based on a change over time of sound reflection characteristics is that testing for the ear health condition can be performed during normal playback of user selected content and without the user's active participation, thus enabling improved ear health without negatively impacting the user's audio experience.

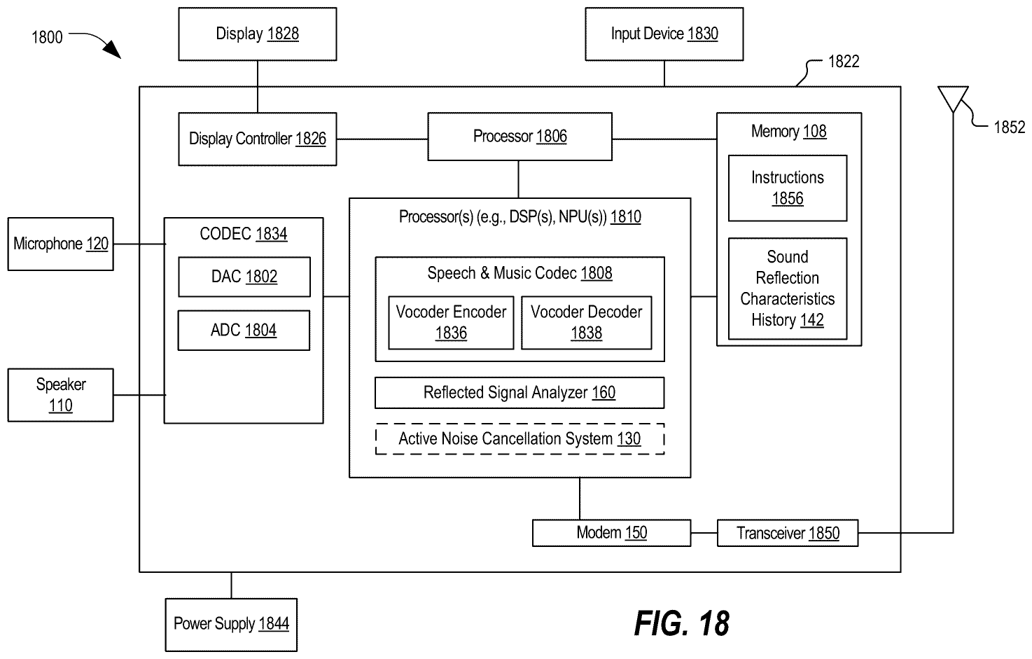

The method 1500 of FIG. 15 may be implemented by a field-programmable gate array (FPGA) device, an application-specific integrated circuit (ASIC), a processing unit such as a central processing unit (CPU), a DSP, a controller, another hardware device, firmware device, or any combination thereof. As an example, the method 1500 of FIG. 15 may be performed by a processor that executes instructions, such as described with reference to FIG. 18.

FIG. 16 depicts a particular implementation of a method 1600 of ear health condition detection. In a particular aspect, one or more operations of the method 1600 are performed by at least one of the active noise cancellation system 130, the reflected signal analyzer 160, the one or more processors 106, the device 102, the system 100, or a combination thereof.

The method 1600 includes, at block 1602, generating, at a speaker of an earphone, an output audio signal. For example, the speaker 110 generates the output audio signal 114 based on the output signal 112.

The method 1600 includes, at block 1604, receiving a reflection of the output audio signal from within an ear canal, such as from an eardrum. For example, the reflection of the output signal can be received at a feedback microphone of the earphone, such as the reflected audio signal 122 from the eardrum 184 that is received at the feedback microphone 120.

The method 1600 includes, at block 1606, receiving a signal from the feedback microphone of the earphone, where the signal received from the feedback microphone includes a component corresponding to the reflection, such as the received signal 124 that includes the second component 286 corresponding to reflection from the eardrum 184.

The method 1600 includes, at block 1608, determining, based on a change over time of sound reflection characteristics represented in the received signal, a condition corresponding to ear wax buildup or water in the ear canal. For example, the reflected signal analyzer 160 determines the ear wax buildup condition 146 or the ear canal fluid condition 254 based on the change over time 144 of the sound reflection characteristics 140.

One benefit of determining a condition corresponding to ear wax buildup is that the user can be notified to take remedial action, resulting in improved hearing, better ear health, and an enhanced user experience, as compared to when the gradual ear wax buildup and the resulting gradual hearing impairment is unnoticed by the user and therefore not corrected. Another benefit of determining the ear wax buildup condition based on sound reflection characteristics is that testing can be performed during normal playback of user selected content and without the user's active participation.

The method 1600 of FIG. 16 may be implemented by a field-programmable gate array (FPGA) device, an application-specific integrated circuit (ASIC), a processing unit such as a central processing unit (CPU), a DSP, a controller, another hardware device, firmware device, or any combination thereof. As an example, the method 1600 of FIG. 16 may be performed by a processor that executes instructions, such as described with reference to FIG. 18.

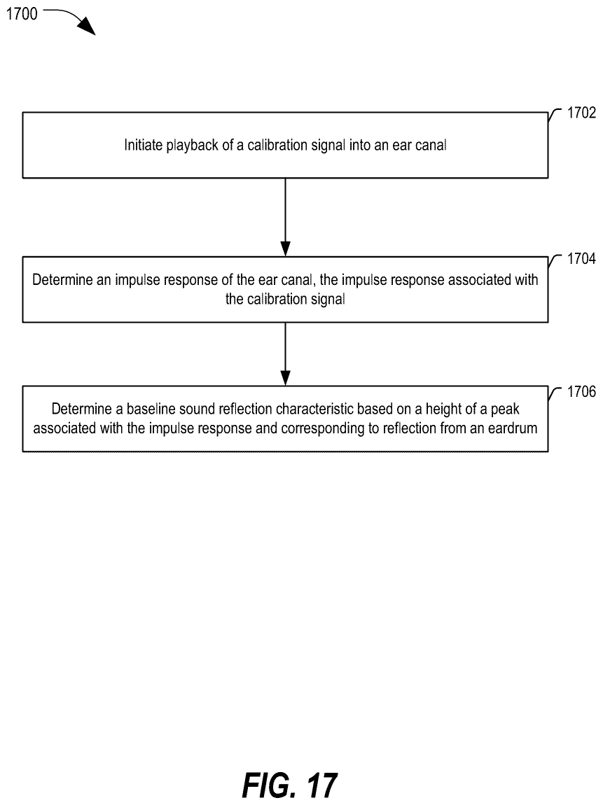

FIG. 17 depicts a particular implementation of a method 1700 of performing a calibration operation that can be used in conjunction with ear health condition detection. For example, the calibration operation of the method 1700 can be included in the method 1500 of FIG. 15, in the method 1600 of FIG. 16, or both. In a particular aspect, one or more operations of the method 1700 are performed by at least one of the active noise cancellation system 130, the reflected signal analyzer 160, the one or more processors 106, the device 102, the system 100, or a combination thereof.

The method 1700 includes, at block 1702, initiating playback of a calibration signal into an ear canal. For example, the device 102 (e.g., the one or more processors 106) performs the calibration operation 260 including initiating playback of the calibration signal 262 as an output signal 112 to generate, at the speaker 110, an output audio signal 114 to the ear canal 186.

The method 1700 includes, at block 1704, determining an impulse response of the ear canal, the impulse response associated with the calibration signal. For example, during the calibration operation 260, the device 102 generates adaptive filter weights 132 that correspond to the impulse response 280 associated with the calibration signal 262 reflected from the eardrum 184.

The method 1700 includes, at block 1706, determining a baseline sound reflection characteristic based on a height of a peak associated with the impulse response and corresponding to reflection from an ear drum, such as described for the baseline sound reflection characteristic 264.

Referring to FIG. 18, a block diagram of a particular illustrative implementation of a device is depicted and generally designated 1800. In various implementations, the device 1800 may have more or fewer components than illustrated in FIG. 18. In an illustrative implementation, the device 1800 may correspond to the device 102. In an illustrative implementation, the device 1800 may perform one or more operations described with reference to FIGS. 1-17.

In a particular implementation, the device 1800 includes a processor 1806 (e.g., a central processing unit (CPU)). The device 1800 may include one or more additional processors 1810 (e.g., one or more DSPs). In a particular aspect, the processor(s) 106 of FIG. 1 corresponds to the processor 1806, the processors 1810, or a combination thereof. The processor(s) 1810 may include a speech and music coder-decoder (CODEC) 1808 that includes a voice coder (“vocoder”) encoder 1836 and a vocoder decoder 1838. In the example illustrated in FIG. 18, the processor(s) 1810 also include the reflected signal analyzer 160 and, optionally, the active noise cancellation system 130.

The device 1800 may include a memory 108 and a CODEC 1834. The memory 108 may include instructions 1856 that are executable by the one or more additional processors 1810 (or the processor 1806) to implement the functionality described with reference to the reflected signal analyzer 160, the active noise cancellation system 130, or a combination thereof. In the example illustrated in FIG. 18, the memory 108 also includes the sound reflection characteristics history 142.

The device 1800 may include a display 1828 coupled to a display controller 1826 and may also include the modem 150 coupled, via a transceiver 1850, to the antenna 1852. One or more speakers 110, one or more microphones including the feedback microphone(s) 120, or both, may be coupled to the CODEC 1834. The CODEC 1834 may include a digital-to-analog converter (DAC) 1802, an analog-to-digital converter (ADC) 1804, or both. In a particular implementation, the CODEC 1834 may receive analog signals from the feedback microphone(s) 120, convert the analog signals to digital signals using the analog-to-digital converter 1804, and provide the digital signals to the speech and music codec 1808. The speech and music codec 1808 may process the digital signals, and the digital signals may further be processed by the active noise cancellation system 130 to determine the adaptive filter weights 132. In a particular implementation, the speech and music codec 1808 may provide digital signals to the CODEC 1834. The CODEC 1834 may convert the digital signals to analog signals using the digital-to-analog converter 1802 and may provide the analog signals to the speaker(s) 110.

In a particular implementation, the device 1800 may be included in a system-in-package or system-on-chip device 1822. In a particular implementation, the memory 108, the processor 1806, the processors 1810, the display controller 1826, the CODEC 1834, and the modem 150 are included in the system-in-package or system-on-chip device 1822. In a particular implementation, an input device 1830 and a power supply 1844 are coupled to the system-in-package or the system-on-chip device 1822. Moreover, in a particular implementation, as illustrated in FIG. 18, the display 1828, the input device 1830, the speaker(s) 110, the microphone(s) 120, an antenna 1852, and the power supply 1844 are external to the system-in-package or the system-on-chip device 1822. In a particular implementation, each of the display 1828, the input device 1830, the speaker(s), the microphone(s) 120, the antenna 1852, and the power supply 1844 may be coupled to a component of the system-in-package or the system-on-chip device 1822, such as an interface or a controller.

The device 1800 may include a smart speaker, a speaker bar, a mobile communication device, a smart phone, a cellular phone, a laptop computer, a computer, a tablet, a personal digital assistant, a display device, a television, a gaming console, a music player, a radio, a digital video player, a digital video disc (DVD) player, a tuner, a camera, a navigation device, a vehicle, a headset, an augmented reality headset, a mixed reality headset, a virtual reality headset, an aerial vehicle, a home automation system, a voice-activated device, a wireless speaker and voice activated device, a portable electronic device, a car, a computing device, a communication device, an internet-of-things (IoT) device, a virtual reality (VR) device, a base station, a mobile device, or any combination thereof.

In conjunction with the described implementations, an apparatus includes means for receiving a signal from a feedback microphone of an earphone. For example, the means for receiving the signal can correspond to the active noise cancellation system 130, the reflected signal analyzer 160, the one or more processors 106, the device 102, the FIR filter 210, the subtracter 212, one or more other circuits or components configured to receive a signal from a feedback microphone of an earphone, or any combination thereof.

In conjunction with the described implementations, the apparatus also includes means for determining, based on a change over time of sound reflection characteristics represented in the received signal, a condition corresponding to ear wax buildup. For example, the means for determining the condition corresponding to ear wax buildup can correspond to the reflected signal analyzer 160, the one or more processors 106, the device 102, one or more other circuits or components configured to determine, based on a change over time of sound reflection characteristics represented in the received signal, a condition corresponding to ear wax buildup, or any combination thereof.

In some implementations, a non-transitory computer-readable medium (e.g., a computer-readable storage device, such as the memory 108) stores instructions (e.g., the instructions 1856) that, when executed by one or more processors (e.g., the one or more processors 106, the one or more processors 1810 or the processor 1806), cause the one or more processors to receive a signal from a feedback microphone of an earphone (e.g., the received signal 124 from the feedback microphone 120 of the earphone 104); and determine, based on a change over time of sound reflection characteristics represented in the received signal, a condition corresponding to ear wax buildup (e.g., the ear wax buildup condition 146 determined based on the change over time 144 of the sound reflection characteristics 140 represented in the received signal 124).

Particular aspects of the disclosure are described below in a set of interrelated Examples:
According to example 1, a device comprises: a processor configured to: receive a signal from a feedback microphone of an earphone; and determine, based on a change over time of sound reflection characteristics represented in the received signal, a condition corresponding to ear wax buildup or water in an ear canal.
Example 2 includes the device of example 1, wherein the processor is configured to track the sound reflection characteristics over time based on adaptive filter weights of an active noise cancellation system.
Example 3 includes the device of example 2, wherein the sound reflection characteristics correspond to a height of a peak, wherein the peak is associated with the adaptive filter weights and corresponds to a reflection from an eardrum.
Example 4 includes the device of any of example 1 to example 3, wherein the processor is integrated in the earphone, and further comprising: a speaker configured to generate an output audio signal; and the feedback microphone, wherein the signal received from the feedback microphone includes a component corresponding to a reflection of the output audio signal from an eardrum.
Example 5 includes the device of any of example 1 to example 4, wherein the earphone corresponds to an in-ear style earphone.
Example 6 includes the device of any of example 1 to example 5, wherein the processor includes a digital signal processor.
Example 7 includes the device of any of example 1 to example 6, wherein the processor is configured to determine the sound reflection characteristics during playback of user-selected audio content at the earphone.
Example 8 includes the device of any of example 1 to example 7, wherein, during a calibration operation, the processor is configured to: initiate playback of a calibration signal into an ear canal; determine an impulse response of the ear canal, the impulse response associated with the calibration signal; and determine a baseline sound reflection characteristic based on a height of a peak associated with the impulse response and corresponding to reflection from an eardrum.
Example 9 includes the device of example 8, wherein the processor is configured to determine the condition corresponding to ear wax buildup based on the height of the peak falling below a threshold.
Example 10 includes the device of any of example 1 to example 9, wherein the processor is configured to send a signal to a user interface device to indicate an audio propagation condition.
Example 11 includes the device of example 10, wherein the audio propagation condition corresponds to at least one of: ear wax buildup, ear tip blockage, ear canal fluid, or ear canal swelling.
According to example 12, a method comprises: receiving, at a processor, a signal from a feedback microphone of an earphone; and determining, based on a change over time of sound reflection characteristics represented in the received signal, a condition corresponding to ear wax buildup or water in an ear canal.
Example 13 includes the method of example 12, further comprising: generating, at a speaker of the earphone, an output audio signal; and receiving a reflection of the output audio signal from an eardrum, wherein the signal received from the feedback microphone includes a component corresponding to the reflection.
Example 14 includes the method of example 12 or example 13, wherein the sound reflection characteristics are determined based on adaptive filter weights of an active noise cancellation system.
Example 15 includes the method of example 14, wherein the sound reflection characteristics correspond to a height of a peak associated with the adaptive filter weights and corresponding to a reflection from an eardrum.
Example 16 includes the method of any of example 12 to example 15, wherein the earphone corresponds to an in-ear style earphone.
Example 17 includes the method of any of example 12 to example 16, wherein the processor includes a digital signal processor.
Example 18 includes the method of any of example 12 to example 17, wherein the sound reflection characteristics are determined during playback of user-selected audio content at the earphone.
Example 19 includes the method of any of example 12 to example 18, further comprising performing a calibration operation including: initiating playback of a calibration signal into an ear canal; determining an impulse response of the ear canal, the impulse response associated with the calibration signal; and determining a baseline sound reflection characteristic based on a height of a peak associated with the impulse response and corresponding to reflection from an ear drum.
Example 20 includes the method of example 19, wherein determining the condition corresponding to ear wax buildup is based on the height of the peak falling below a threshold.
Example 21 includes the method of any of example 12 to example 20, wherein the processor is configured to send a signal to a user interface device to indicate an audio propagation condition.
Example 22 includes the method of example 21, wherein the audio propagation condition corresponds to at least one of: ear wax buildup, ear tip blockage, ear canal fluid, or ear canal swelling.
According to example 23, a non-transitory computer-readable medium storing instructions that, when executed by one or more processors, cause the one or more processors to: receive a signal from a feedback microphone of an earphone; and determine, based on a change over time of sound reflection characteristics represented in the received signal, a condition corresponding to ear wax buildup or water in an ear canal.
Example 24 includes the non-transitory computer-readable medium of example 23, wherein the instructions further cause the one or more processors to: generate, at a speaker of the earphone, an output audio signal; and receive a reflection of the output audio signal from an eardrum, wherein the signal received from the feedback microphone includes a component corresponding to the reflection.
Example 25 includes the non-transitory computer-readable medium of example 23 or example 24, wherein the sound reflection characteristics are determined based on adaptive filter weights of an active noise cancellation system.
Example 26 includes the non-transitory computer-readable medium of example 25, wherein the sound reflection characteristics correspond to a height of a peak associated with the adaptive filter weights and corresponding to a reflection from an eardrum.
Example 27 includes the non-transitory computer-readable medium of any of example 23 to example 26, wherein the earphone corresponds to an in-ear style earphone.
Example 28 includes the non-transitory computer-readable medium of any of example 23 to example 27, wherein the one or more processors includes a digital signal processor.
Example 29 includes the non-transitory computer-readable medium of any of example 23 to example 28, wherein the sound reflection characteristics are determined during playback of user-selected audio content at the earphone.
Example 30 includes the non-transitory computer-readable medium of any of example 23 to example 29, wherein the instructions further cause the one or more processors to perform a calibration operation including: initiating playback of a calibration signal into an ear canal; determining an impulse response of the ear canal, the impulse response associated with the calibration signal; and determining a baseline sound reflection characteristic based on a height of a peak associated with the impulse response and corresponding to reflection from an ear drum.
Example 31 includes the non-transitory computer-readable medium of example 30, wherein determining the condition corresponding to ear wax buildup is based on the height of the peak falling below a threshold.
Example 32 includes the non-transitory computer-readable medium of any of example 23 to example 31, wherein the instructions further cause the one or more processors to send a signal to a user interface device to indicate an audio propagation condition.
Example 33 includes the non-transitory computer-readable medium of example 32, wherein the audio propagation condition corresponds to at least one of: ear wax buildup, ear tip blockage, ear canal fluid, or ear canal swelling.
According to example 34, an apparatus comprises: means for receiving a signal from a feedback microphone of an earphone; and means for determining, based on a change over time of sound reflection characteristics represented in the received signal, a condition corresponding to ear wax buildup or water in an ear canal.
Example 35 includes the apparatus of example 34, further comprising means for generating an output audio signal, and wherein the signal received from the feedback microphone includes a component corresponding to a reflection of the output audio signal from an eardrum.
Example 36 includes the apparatus of example 34 or example 35, wherein the sound reflection characteristics are determined based on adaptive filter weights of an active noise cancellation system.
Example 37 includes the apparatus of example 36, wherein the sound reflection characteristics correspond to a height of a peak associated with the adaptive filter weights and corresponding to a reflection from an eardrum.
Example 38 includes the apparatus of any of example 34 to example 37, wherein the earphone corresponds to an in-ear style earphone.
Example 39 includes the apparatus of any of example 34 to example 38, wherein the means for determining includes a digital signal processor.
Example 40 includes the apparatus of any of example 34 to example 39, wherein the sound reflection characteristics are determined during playback of user-selected audio content at the earphone.
Example 41 includes the apparatus of any of example 34 to example 40, further comprising means for performing a calibration operation including: means for initiating playback of a calibration signal into an ear canal; means for determining an impulse response of the ear canal, the impulse response associated with the calibration signal; and means for determining a baseline sound reflection characteristic based on a height of a peak associated with the impulse response and corresponding to reflection from an ear drum.
Example 42 includes the apparatus of example 41, wherein determining the condition corresponding to ear wax buildup is based on the height of the peak falling below a threshold.
Example 43 includes the apparatus of any of example 34 to example 42, further comprising means for sending a signal to a user interface device to indicate an audio propagation condition.
Example 44 includes the apparatus of example 43, wherein the audio propagation condition corresponds to at least one of: ear wax buildup, ear tip blockage, ear canal fluid, or ear canal swelling.

Those of skill would further appreciate that the various illustrative logical blocks, configurations, modules, circuits, and algorithm steps described in connection with the implementations disclosed herein may be implemented as electronic hardware, computer software executed by a processor, or combinations of both. Various illustrative components, blocks, configurations, modules, circuits, and steps have been described above generally in terms of their functionality. Whether such functionality is implemented as hardware or processor executable instructions depends upon the particular application and design constraints imposed on the overall system. Skilled artisans may implement the described functionality in varying ways for each particular application, such implementation decisions are not to be interpreted as causing a departure from the scope of the present disclosure.

The steps of a method or algorithm described in connection with the implementations disclosed herein may be embodied directly in hardware, in a software module executed by a processor, or in a combination of the two. A software module may reside in random access memory (RAM), flash memory, read-only memory (ROM), programmable read-only memory (PROM), erasable programmable read-only memory (EPROM), electrically erasable programmable read-only memory (EEPROM), registers, hard disk, a removable disk, a compact disc read-only memory (CD-ROM), or any other form of non-transient storage medium known in the art. An exemplary storage medium is coupled to the processor such that the processor may read information from, and write information to, the storage medium. In the alternative, the storage medium may be integral to the processor. The processor and the storage medium may reside in an application-specific integrated circuit (ASIC). The ASIC may reside in a computing device or a user terminal. In the alternative, the processor and the storage medium may reside as discrete components in a computing device or user terminal.

The previous description of the disclosed aspects is provided to enable a person skilled in the art to make or use the disclosed aspects. Various modifications to these aspects will be readily apparent to those skilled in the art, and the principles defined herein may be applied to other aspects without departing from the scope of the disclosure. Thus, the present disclosure is not intended to be limited to the aspects shown herein but is to be accorded the widest scope possible consistent with the principles and novel features as defined by the following claims.

What is claimed is:

1. A device comprising:
a processor configured to:
receive a signal from a feedback microphone of an earphone; and
determine, based on a change over time of sound reflection characteristics represented in the received signal, a condition corresponding to ear wax buildup or water in an ear canal.

2. The device of claim 1, wherein the processor is configured to track the sound reflection characteristics over time based on adaptive filter weights of an active noise cancellation system.

3. The device of claim 2, wherein the sound reflection characteristics correspond to a height of a peak associated with the adaptive filter weights and corresponding to a reflection from an eardrum.

4. The device of claim 1, wherein the processor is integrated in the earphone, and further comprising:
a speaker configured to generate an output audio signal; and
the feedback microphone, wherein the signal received from the feedback microphone includes a component corresponding to a reflection of the output audio signal from an eardrum.

5. The device of claim 4, wherein the earphone corresponds to an in-ear style earphone.

6. The device of claim 4, wherein the processor includes a digital signal processor.

7. The device of claim 1, wherein the processor is configured to determine the sound reflection characteristics during playback of user-selected audio content at the earphone.

8. The device of claim 1, wherein, during a calibration operation, the processor is configured to:
initiate playback of a calibration signal into the ear canal;
determine an impulse response of the ear canal, the impulse response associated with the calibration signal; and
determine a baseline sound reflection characteristic based on a height of a peak associated with the impulse response and corresponding to reflection from an eardrum.

9. The device of claim 8, wherein the processor is configured to determine the condition corresponding to ear wax buildup based on the height of the peak falling below a threshold.

10. The device of claim 1, wherein the processor is configured to send a signal to a user interface device to indicate an audio propagation condition.

11. The device of claim 10, wherein the audio propagation condition corresponds to at least one of: ear wax buildup, ear tip blockage, ear canal fluid, or ear canal swelling.

12. A method comprising:
receiving, at a processor, a signal from a feedback microphone of an earphone; and
determining, based on a change over time of sound reflection characteristics represented in the received signal, a condition corresponding to ear wax buildup or water in an ear canal.

13. The method of claim 12, further comprising:
generating, at a speaker of the earphone, an output audio signal; and
receiving a reflection of the output audio signal from an eardrum, wherein the signal received from the feedback microphone includes a component corresponding to the reflection.

14. The method of claim 12, wherein the sound reflection characteristics are determined based on adaptive filter weights of an active noise cancellation system.

15. The method of claim 14, wherein the sound reflection characteristics correspond to a height of a peak associated with the adaptive filter weights and corresponding to a reflection from an eardrum.

16. The method of claim 12, wherein the sound reflection characteristics are determined during playback of user-selected audio content at the earphone.

17. The method of claim 12, further comprising performing a calibration operation including:
initiating playback of a calibration signal into the ear canal;
determining an impulse response of the ear canal, the impulse response associated with the calibration signal; and
determining a baseline sound reflection characteristic based on a height of a peak associated with the impulse response and corresponding to reflection from an ear drum.

18. The method of claim 17, wherein determining the condition corresponding to ear wax buildup is based on the height of the peak falling below a threshold.

19. A non-transitory computer-readable medium storing instructions that, when executed by one or more processors, cause the one or more processors to:
receive a signal from a feedback microphone of an earphone; and
determine, based on a change over time of sound reflection characteristics represented in the received signal, a condition corresponding to ear wax buildup or water in an ear canal.

20. The non-transitory computer-readable medium of claim 19, wherein the sound reflection characteristics are determined based on adaptive filter weights of an active noise cancellation system.
